(19) United States
(12) Patent Application Publication
(10) Pub. No.: US 2009/0220583 A1
(43) Pub. Date: Sep. 3, 2009

(54) Method and composition for treating inflammatory disorders

(76) Inventors: Lena Pereswetoff-Morath, Solna (SE); Anders Carlsson, Stockholm (SE)

Correspondence Address: MORGAN LEWIS & BOCKIUS LLP, 1111 PENNSYLVANIA AVENUE NW, WASHINGTON, DC, 20004, US

(21) Appl. No.: 11/921,850

(22) PCT Filed: Jun. 8, 2006
(86) PCT No.: PCT/GB2006/002090

§ 371 Date: Apr. 10, 2009

Related U.S. Application Data
(60) Provisional application No. 60/688,698, filed on Jun. 9, 2005.
(60) Provisional application No. 60/696,777, filed on Jul. 7, 2005.

(51) Int. Cl. A61K 9/127; A61K 31/58; A61K 31/56; A61K 31/381; A61K 31/55; A61K 31/47; A61K 31/444; A61P 29/00

(52) U.S. Cl. 424/450; 514/174; 514/178; 514/443; 514/217.05; 514/311; 514/334; 977/907

(57) ABSTRACT

There is provided homogeneous pharmaceutical compositions for the treatment of inflammatory disorders comprising an antiinflammatory and/or antihistaminic active ingredient, a polar lipid liposome and a pharmaceutically-acceptable aqueous carrier.

### FIELD OF THE INVENTION

This invention relates to compositions for use in methods of treating inflammatory disorders, and to processes for their preparation.

### BACKGROUND AND PRIOR ART

There are many diseases/disorders that are inflammatory in their nature. Inflammatory diseases that affect the population include asthma, inflammatory bowel disease, rheumatoid arthritis, osteoarthritis, rhinitis, conjunctivitis and dermatitis.

Inflammation is also a common cause of pain. Inflammatory pain may arise for numerous reasons, such as infection, surgery or other trauma. Moreover, several diseases including malignancies and cardiovascular diseases are known to have inflammatory components adding to the symptomatology of the patients.

Asthma is a disease of the airways that contains elements of both inflammation and bronchoconstriction. Treatment regimens for asthma are based on the severity of the condition. Mild cases are either untreated or are only treated with inhaled β-agonists which affect the bronchoconstriction element, whereas patients with more severe asthma are typically treated regularly with inhaled corticosteroids which to a large extent are antiinflammatory in their nature. A new preventative therapy for asthma works by blocking the production of proinflammatory leukotrienes and cytokines through inhibiting the 5-lipoxygenase enzyme.

Allergic and non-allergic rhinitis are common disorders affecting about 30% of the population Rhinitis has a considerable impact on quality of life. If fact, rhinitis is regarded to affect the quality of life more so than, e.g., asthma.

Hay fever and perennial allergic rhinitis are characterised by sneezing, rhinorrhea, nasal congestion, pruritus, conjunctivitis and pharyngitis. In perennial rhinitis, chronic nasal obstruction is often prominent and may extend to eustachian tube obstruction.

Oral or local antihistamines are first line treatments, and nasal steroids second line treatments for rhinitis. For most patients, topical corticosteroids and long acting antihistamine agents provide significant relief of symptoms. Antihistamines may also affect non-immunologically (non-IgE) mediated hypersensitivity reactions such as non-allergic rhinitis, exercise induced asthma, cold urticaria, and non-specific bronchial hyperreactivity.

The main clinical effects of antihistamines include reduced sneezing and rhinorrhea. However, nasal blockage appears to be less responsive. Local administration of antihistamines (such as azelastine and levocabastine) has advantages, including rapid onset of action and fewer side effects.

Inflammatory pain may be reduced by the inhibition of the cyclooxygenase (COX) enzyme. The COX enzyme exists in two forms, one that is constitutively expressed in many cells and tissues (COX-1), and one that is induced by proinflammatory stimuli, such as cytokines, during an inflammatory response (COX-2).

COXs metabolise arachidonic acid to the unstable intermediate prostaglandin H₂ (PGH₂). PGH₂ is further metabolized to other prostaglandins including PGE₂, PGF2α, PGD₂, prostacyclin and thromboxane A₂. These arachidonic acid metabolites are known to have pronounced physiological and pathophysiological activity including proinflammatory effects.

PGE₂ in particular is known to be a strong proinflammatory mediator, and is also known to induce fever and pain. Consequently, numerous drugs have been developed with a view to inhibiting the formation of PGE₂, including “NSAIDs” (non-steroidal antiinflammatory drugs) and “coxibs” (selective COX-2 inhibitors). These drugs act predominantly by inhibition of COX-1 and/or COX-2, thereby reducing the formation of PGE₂.

The leukotrienes (LTs) are formed from arachidonic acid by a set of enzymes distinct from those in the COX/PGES pathway. The key enzyme in leukotriene biosynthesis is 5-lipoxygenase (5-LO), which in a two-step reaction catalyzes the formation of LTA₄ from arachidonic acid. Leukotriene A₄ can be further metabolized into Leukotriene B₄, a reaction catalyzed by LTA₄ hydrolase. Cellular leukotriene biosynthesis is dependent on 5-lipoxygenase activating protein (FLAP), a membrane bound protein which binds arachidonic acid and facilitates the 5-lipoxygenase reaction. Leukotriene B₄ is known to be a strong proinflammatory mediator, while the cysteinyl-containing leukotrienes C₄, D₄ and E₄ (CysLTs) are very potent bronchoconstrictors and proinflammatory mediators that have been implicated in the pathobiology of asthma and inflammation. Therefore, the marketed 5-LO inhibitors and antagonists of cysteinyl-containing leukotriene receptors 1 and 2 represent two new classes of anti-inflammatory treatments, while the development of marketed FLAP inhibitors, leukotriene A₄ hydrolase inhibitors, leukotriene B₄ receptor antagonists may provide further new classes of anti-inflammatory treatments.

Phosphodiesterase type 4 (PDE 4) plays an important role in modulating the activity of cells that are involved in the inflammatory processes that occur in chronic obstructive pulmonary disorder (COPD) and asthma. PDE4 inhibitors represent a new class of drugs that have the potential to inhibit bronchoconstriction as well as inhibit inflammatory cell activity (including inhibiting the production of leukotrienes).

Liposomes (also known as lipid vesicles) are colloidal particles that are prepared from polar lipid molecules derived either from natural sources or chemical synthesis. Such spherical, closed structures composed of curved lipid bilayers, are typically used to entrap drugs, which are often cytotoxic, in order to reduce toxicity and/or increase efficacy. Liposome-entrapped drug preparations are often provided in a dry (e.g. freeze-dried) form, which is subsequently reconstituted with an aqueous solution immediately prior to administration. This is done in order to minimise the possibility of leakage of e.g. cytotoxic drug into aqueous solution and thereby reducing the entrapping effect of the liposome.

Liposomes have also been employed to encapsulate various drug compounds for delivery via the nasal route, in order to improve bioavailability or as an adjuvant. Drugs that may be mentioned include tetanus toxoid vaccine, insulin, desmopressin and diphenhydramine hydrochloride (see Türker et al, Review Article: Nasal Route and Drug Delivery Systems, Pharm. World Sci., 2004; 26, 137-142 and the references cited therein), as well as ciprofloxacin, CM3 and salbutamol (see Desai et al, A Facile Method of Delivery of Ltposomes by Nebulization, J. Control. Release, 2002; 84, 69-78).

Liposome-entrapped cetirizine has been administered topically to evaluate peripheral antihistaminic activity and systemic absorption in a rabbit model (Elzainy et al, Cetirizine from Topical Phosphatidylcholine-Hydrogenated Liposomes, The AAPS Journal, 2004; 6, 1-7, see also Drug Development and Industrial Pharmacy, 2005; 31, 281-291).

Homogeneous pharmaceutical compositions containing cede and a polar lipid liposome have been disclosed in international patent application WO 2005/107711.

The lipophilic behaviour of cetirizine in buffered aqueous phosphatidylcholine liposome systems has also been studied (Plemper van Balen G et al., Lipophilicity behaviour of the zwitterionic antihistamine cetirizine in phosphatidyicholine liposomes/water systems, Pharm. Res. 2001; 18, 694-701).

Examples of other formulations comprising inter alia liposome-encapsulated active ingredients are discussed in U.S. Pat. No. 4,427,649, U.S. Pat. No. 5,569,464, EP 0249561, WO 00/38681, U.S. Pat. No. 4,839,175 and WO 98/00111.

Surprisingly, we have found that the irritation that may be associated with (e.g. nasal) administration of certain antiinflammatory and/or antihistaminic active ingredients may be reduced by way of use of a homogeneous pharmaceutical compositions comprising such active ingredients, a polar lipid liposome and a pharmaceutically acceptable carrier.

According to the invention, there is provided a homogeneous pharmaceutical composition suitable for the treatment of an inflammatory disorder comprising an antiinflammatory and/or antihistaminic active ingredient, a polar lipid liposome and a pharmaceutically-acceptable aqueous carrier, provided that the active ingredient is not cetirizine, which compositions are referred to hereinafter as “the compositions of the invention”.

The skilled person will appreciate that antiinflammatory and/or antihistaminic active ingredients are employed in compositions of the invention in a pharmacologically-effective amount (vide infra). The term “pharmacologically-effective amount” refers to an amount of the antiinflammatory and/or antihistaminic active ingredient, which is capable of conferring the desired therapeutic effect on a treated patient, whether administered alone or in combination with another active ingredient. Such an effect may be objective (i.e. measurable by some test or marker) or subjective (i.e. the subject gives an indication of, or feels, an effect).

By “pharmaceutical compositions” we include compositions that are suitable for use in direct administration to mammals, and especially humans. In this respect, the term is intended to encompass formulations that include only components that are regarded in the art as suitable for administration to mammalian, and especially human, patients. In the context of the present invention, the term may also mean that the compositions of the invention are in a form of a liquid that is ready-to-use, directly from the shelf, and not a formulation in which drug is encapsulated inside liposomes requiring reconstitution shortly prior to administration in order to avoid leakage of drug from liposomes into an aqueous carrier.

By “homogeneous” we include not only that the compositions of the invention comprise liposomes dispersed evenly throughout the aqueous carrier, but further that the active ingredient is distributed throughout the whole composition. This means that, following formation of a mixture comprising liposomes and drug in aqueous medium, drug that is not encapsulated within liposome is not removed following liposome formation. This may, in the case of certain compositions of the invention, result in a substantially similar concentration of active ingredient in the relevant aqueous medium, whether that medium is located inside or outside of the liposomal structures. By “substantially similar”, we include that the concentration may vary by about ±50%, such as about ±40%, preferably about ±30%, more preferably about ±20% and particularly about ±10% (when comparing concentrations inside and outside of the liposomal structures) at room temperature and atmospheric pressure. Drug concentration profiles may be measured by standard techniques known to the skilled person, such as ³¹P-NMR. For example, a standard in situ probing technique, or a technique that involves separation of the liposomal fraction from the free aqueous carrier and measurement of the amount/concentration of drug associated with each fraction may be employed. Separation may be accomplished by centrifugation, dialysis, ultrafiltration, or gel filtration.

It is preferred that the compositions of the invention further include a pharmaceutically-acceptable buffer capable of providing a pH of from about pH 4 (e.g. 4.0) to about pH 8 (e.g. 8.0), preferably from about pH 5 (e.g. 5.0) to about pH 7 (e.g. 7.0). Appropriate buffers include those that will not interfere with the formation of liposomes, such as a phosphate (e.g. disodium phosphate, dipotassium phosphate, sodium dihydrogen phosphate, potassium dihydrogen phosphate or phosphoric acid plus base), citrate (e.g. sodium citrate or citric acid plus base), or acetate buffer (e.g. sodium acetate or acetic acid plus base), which is capable of maintaining a pH within the above-specified ranges. Buffers may be employed in an amount that is suitable to provide for the above-mentioned effects and such will be appreciated by the skilled person without recourse to inventive input. Appropriate quantities are for example in the range of about 1 mg/mL to about 30 mg/mL.

The term “inflammatory disorder” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Accordingly, compositions of the invention may be useful in the treatment of asthma, chronic obstructive pulmonary disease, pulmonary fibrosis, inflammatory bowel disease, irritable bowel syndrome, inflammatory pain, fever, migraine, headache, low back pain, fibromyalgia, myofascial disorders, viral infections (e.g. influenza, common cold, herpes zoster, hepatitis C and AIDS), bacterial infections, fungal infections, dysmenorrhea, burns, surgical or dental procedures, malignancies (e.g. breast cancer, colon cancer, and prostate cancer), hyperprostaglandin E syndrome, classic Bartter syndrome, atherosclerosis, gout, arthritis, osteoarthritis, juvenile arthritis, rheumatoid arthritis, fever, ankylosing spondylitis, Hodgdin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, neurodegenerative disorders such as Alzheimer's disease and multiple sclerosis, autoimmune diseases, allergic disorders, rhinitis, ulcers, coronary heart disease, sarcoidosis and any other disease with an inflammatory component.

Compositions of the invention find particular utility in the treatment of rhinitis, migraine, acute pain, chronic pain and asthma. The term “rhinitis” will be understood to include any irritation and/or inflammation of the nose, whether allergic or non-allergic, including seasonal rhinitis (e.g. caused by outdoor agents such as pollen; hay fever) and/or perennial rhinitis (e.g. caused by house dust mites, indoor mold etc), as well as the symptoms thereof.

The term “antiinflammatory and/or antihistaminic active ingredient” will be understood by the skilled person to include any substance, whether naturally-occurring or synthetic, with antiinflammatory and/or antihistaminic properties as appropriate. The antiinflammatory class of compounds comprises steroidal anti-inflammatory drugs (corticosteroids) and non-steroidal antiinflammatory drugs (NSAIDs), which latter term includes COX inhibitors, PDE4 inhibitors and leukotriene modifiers (e.g. S-lipoxygenase inhibitors, inhibitors of FLAP, LTA₄ hydrolase inhibitors, LTB₄ receptor antagonists and CysLT (i.e. CysLT1 and CysLT2) receptor antagonists) while the antihistamine class comprises H₁ receptor antagonists. In the context of this invention the term “antiinflammatory and/or antihistaminic active ingredient” also includes anti-migraine compounds, opioids and analogues thereof.

Preferred active ingredients in the compositions of the invention include antihistaminic active ingredients, corticosteroids and leukotriene modifiers.

Anti-migraine compounds that may be mentioned include almotriptan, alpiropride, dihydroergotamine, eletriptan, ergotamine, feverfew, frovatriptan, iprazochrome, methysergide, naratriptan, pizotifen, rizatriptan, sumatriptan, zolmitriptan and commonly employed salts thereof.

Opioids and analogues thereof that may be mentioned include alfentanil, anileridine, bezitramide, buprenorphine, butorphanol, carfentanil, codeine, dextromoramide, dextropropoxyphene, dezocine, diamorphine, dihydrocodeine, dipipanone, embutramide, ethoheptazine, ethylmorphine, etorphine, fentanyl, hydrocodone, hydromorphone, ketobemidone, levacetylmethadol, levomethadone, levophanol, lofexidine, meptszinol, methadone, morphine, nalbuphine, naltrexone, nicomorphine, opium, oxycodone, oxymorphone, papaveretum, pentazocine, pethidine, phenazocine, phenoperidine, pholcodine, piritramide, remifentanil, sufentanil, tilidine, tramadol and commonly employed salts thereof.

Steroidal antiinflammatory compounds that may be mentioned include alclometasone, beclometasone, betamethasone, budesonide, ciclesonide, clobetasol, clobetasone, deflazacort, dexamethasone, diflucortolone valerate, fluocinolone acetonide, fluocinonide, fluocortolone, fluprednidene, fluorometholone, fluticasone, halcinonide, hydrocortisone, methylprednisolone, mometasone, prednisolone, rimexolone and triamcinolone and commonly employed salts thereof. Preferred steroidal anti-inflammatory compounds include budesonide and fluticasone (e.g. the latter in the form of a salt, such as a propionate salt).

NSAIDs (including COX inhibitors) that may be mentioned include aceclofenac, acemetacin, acetanilide, alclofenac, alminoprofen, aloxiprin, aminophenazone, aminopropylone, ampiroxicam, amtolmetin guacil, amyl salicylate, aspirin, azapropazone, bendazac, benoxaprofen, benzydamine, beta-aminopropionitrile, bornyl salicylate, bromofenac, bufexamac, bumadizone, butibufen, carbasalate, carprofen, celecoxib, clofexamide, clofezone, clonixin, dexketoprofen, diclofenac, diflunisal, dipyrone, droxicam, eltenac, epirizole, etodolac, ethenzamide, ethyl salicylate, etofenamate, etoricoxib, felbinac, fenbufen, fenoprofen, fentiazac, fepradinol, feprazone, floctafenine, flufenamic acid, flunixin, flunoxaprofen, flurbiprofen, fosfosal, furprofen, glafenine, glucametacin, glycol salicylate, ibuprofen, ibuproxam, indometacin, ketoprofen, ketorolac, lysine aspirin, mefenamic acid, meloxicam, methyl buteneisalicylate, methyl salicylate, nabumetone, naproxen, nedocromil, nifenazone, niflumic acid, nimesulide, oxaprozin, oxyphenbutazone, paracetamol, parecoxib, phenacetin, phenazone, phenylbutazone, picolamine salicylate, piketoprofen, piroxicam, pranoprofen, proglumetacin propacetamol, propyphenazone, proquazone, ramifenazone, rofecoxib, salamidacetic acid, salicylamide, salix, salol, salsalate, sodium cromoglycate, salicylate, thiosalicylate, sulindac, suprofen, suxibuzone, tenidap, tenoxicam, tetridamine, thurfyl salicylate, tiaprofenic acid, tiaramide, tinoridine, tolfenamic acid, tolmetin, trisalicylate, trolamine salicylate, ufenamate, valdecoxib, vedaprofen and zaltoprofen and commonly employed salts thereof.

Specific inhibitors of PDE4 that may be mentioned include cilomilast, roflumilast, tetomilast, piclarmilast, as well as
(aa) CP-671305, (+)-2-[4-({[benzo[1,3]dioxol-5-yloxy)-pyridine-3-carbonyl]-amino}-methyl)-3-fluoro-phenoxy]-propionic acid,
(bb) SCH351591, N-(3,5-dichloro-1-oxido-pyridinyl)-8-methoxy-2-(trifluoromethyl)-5-quinolinecarboxamide,
(cc) KF 19514, 5-phenyl-3-(3-pyridyl)methyl-3H-imidazo(4,5-c)(1,8)-naphthyridin-4(5H)-one,
(dd) AWD 12-281, N-(3,5-dichloropyrid-4-yl)-(1-(4-fluorobenzyl)-5-hydroxy-indole-3-yl)glyoxylic acid amide,
(ee) D 22888, 1-ethyl-8-methoxy-3-methyl-5-propylimidazo(1,5-a)pyrido(3,2-e)pyrazinone,
(ff) YM976, 4-(3-chlorophenyl)-1,7-diethylpyrido[2,3-d]pyrimidin-2(1H)-one,
(gg) NVP-ABE171, 4-(8-benzo[1,2,5]oxadiazol-5-yl[1,7]naphthyridin-6-yl)-benzoic acid,
(hh) CI-1044, N-(9-amino-4-oxo-1-phenyl-3,4,6,7-tetrahydro(1,4)diazepino-(6,7,1-hi)indol-3-yl)nicotiamide),
(ii) SB 207499, c-4-cyano-4-(3-cyclopentyloxy-4-methoxyphenyl)-1-cyclohexanecarboxylic acid,
(jj) CC-100004, YM-64227, BAY 19-8004 and GRC 3886,
   and commonly employed salts thereof.

CysLT1 and CysLT2 receptor antagonists that may be mentioned include abulukast, cinalukast, iralulast, montelukast, pobilukast, pranlukast, sululast, tomelukast, verlukast, zafirlukast,
(I) BAY-u9773

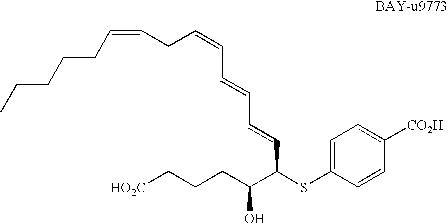

(II) MK571

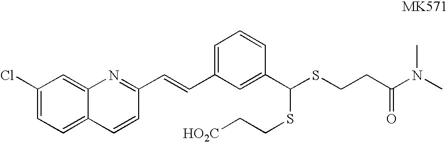

and commonly employed salts thereof. Preferred Cys LT receptor antagonists that may be mentioned include montelukast.

5-lipoxygenase inhibitors that may be mentioned include the following.
(1) Zileuton (synonyms: A-64077, ABT 077, Zyflo®), described in, for example, EP 0 279 263, U.S. Pat. No. 4,873,259, Int. J. Immunopharmacol. 14, 505 (1992), Br. J. Cancer 74, 683 (1996) and Am J. Resp. Critical Care Med. 157, Part 2, 1187 (1998).

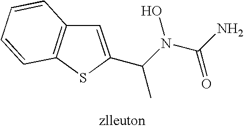

(2) A-63162, described in, for example, Anticancer Res. 14, 1951 (1994).

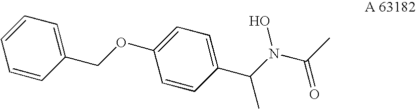

(3) A-72694.

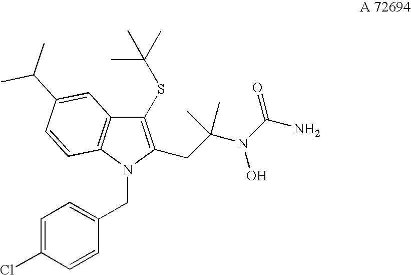

(4) A-78773, described in, for example, Curr. Opin. Invest. Drugs 2, 69 (1993).

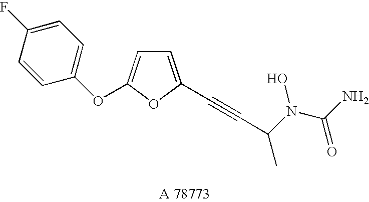

(5) A-79175 (the R-enantiomer of A 78773), described in, for example, Carcinogenesis 19, 1393 (1998) and J. Med. Chem. 40, 1955 (1997).

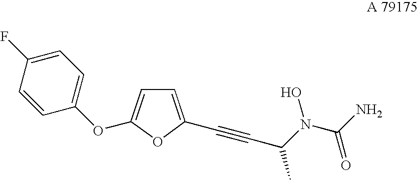

(6) A-80263.

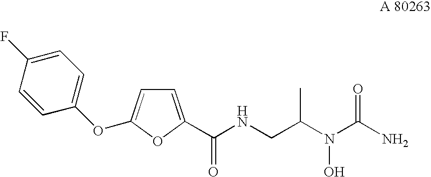

(7) A-81834.

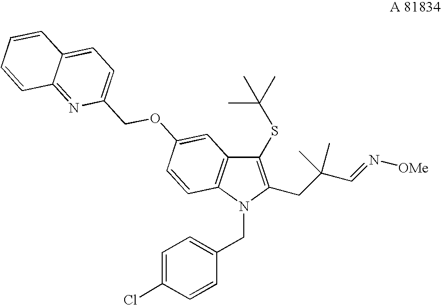

(8) A-93178

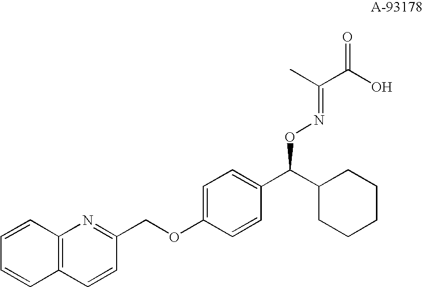

(9) A-121798, described in, for example, 211th Am. Chem. Soc. Meeting. 211: abstr. 246, 24 March 1996.
(10) Atreleuton (synonyms ABT-761 and A-85761), described in, for example, Exp. Opin. Therap. Patents 5 127 (1995).

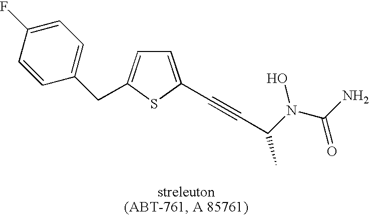

(11) MLN-977 (synonyms LPD-977 and CMI-977), described in, for example, Curr. Opin. Anti-Inflamm. & Immunomod Invest. Drugs 1, 468 (1999). This, as well as similar compounds are described in U.S. Pat. No. 5,703,093.

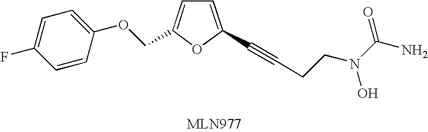

(12) CMI-947, described in, for example, 215th Am. Chem. Soc. Meeting. 215: abstr. MEDI 004, 29 Mar. 1998. This, as well as similar compounds are described in U.S. Pat. No. 5,792,776.

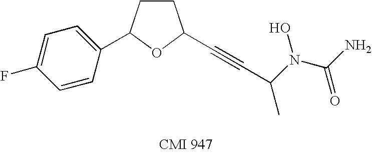

(13) CMI-568, described in, for example, 211th Am. Chem. Soc. Meeting. 211: abstr. 205, 24 Mar. 1996.

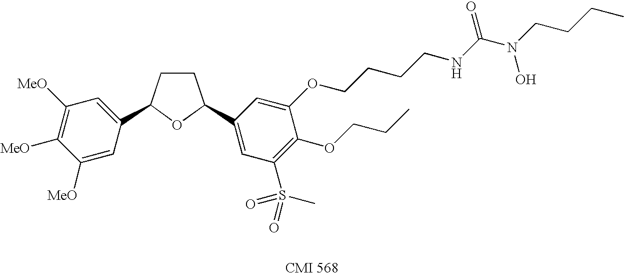

(14) LDP 392 (synonym CMI 392), described in, for example, Pharmacol. Res. 44, 213 (2001).

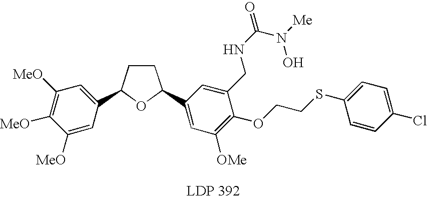

(15) Linetastuie (synonyms: linazolast, TMK 688, YM 257), described in, for example, Int. J. Immunopharmacol. 22, 123 (2000).

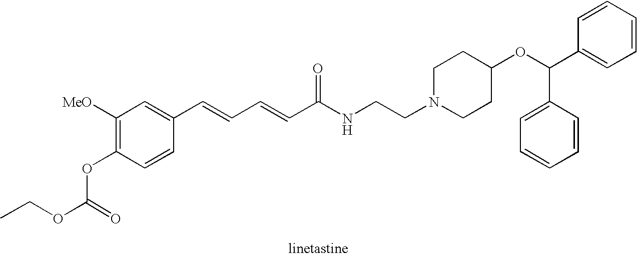

(16) Lonapalene (synonym: RS 43179), described in, for example, Pharm. Res. 9, 1145 (1992).

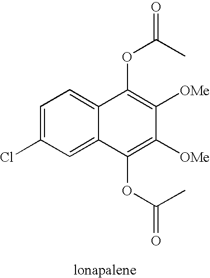

(17) LY-221068, described in, for example, Ann. N.Y. Acad. Sci. (Immunosuppressive and Antiinflammatory Drugs) 696, 415 (1993).

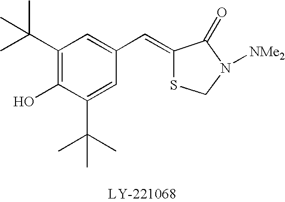

(18) LY 269415, described in, for example, Agents and Actions 42, 67 (1994).

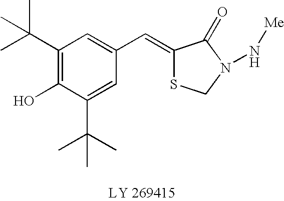

(19) 5-LO inhibitors with histamine H₁ receptor antagonist activity described in, for example, Bioorg. Med. Chem. Lett. 14, 2265 (2004), such as the following compound.

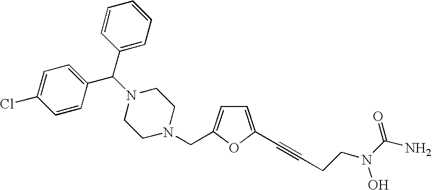

(20) BF-389

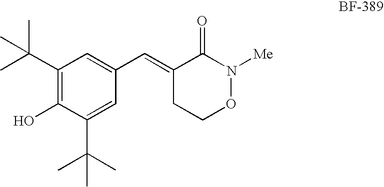

(21) BIL 226 and BIL 357, described in, for example, J. Pharmacol. Exp. Therap. 265, 483 (1993).

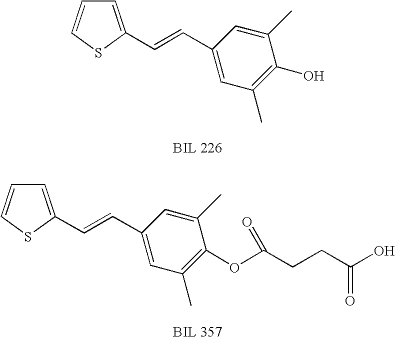

(22) BU 4601A, BU 4601B and BU 4601C, described in, for example, J. Antibiotics 46, 705 (1993).

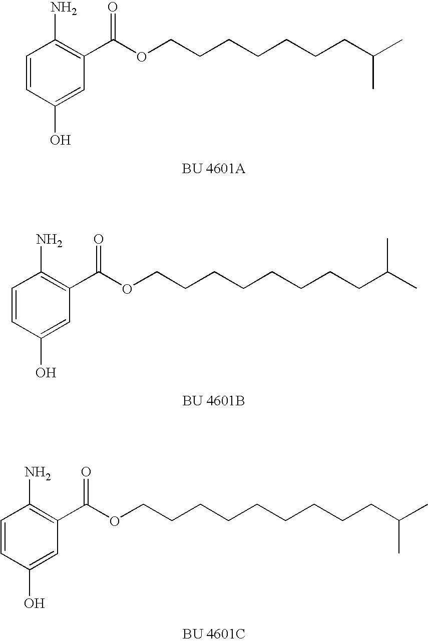

(23) BW 755C, described in, for example, J. Pharm. Therap. Therap. 277, 17(1996).

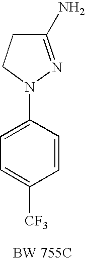

(24) BW-A4C, described in, for example, Eur. J. Biochem. 267, 3633 (2000).

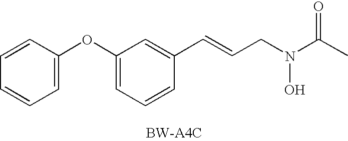

(25) BWB 70C, described in, for example, Br. J. Phamacol. 108 (Suppl), 186P (1993).

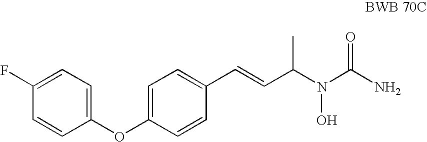

(26) CBS 1108.

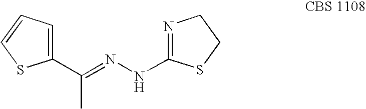

(27) CGS 26529, described in, for example, Inflamm. Res. 44 (Suppl. 2) 147 (1995).

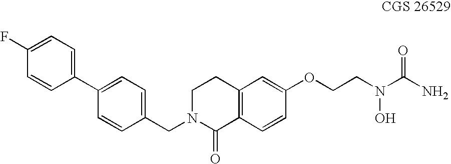

(28) CGS 25667, CGS 25997 and CGS 25998, described in, for example, J. Med. Chem. 38, 68 (1995).

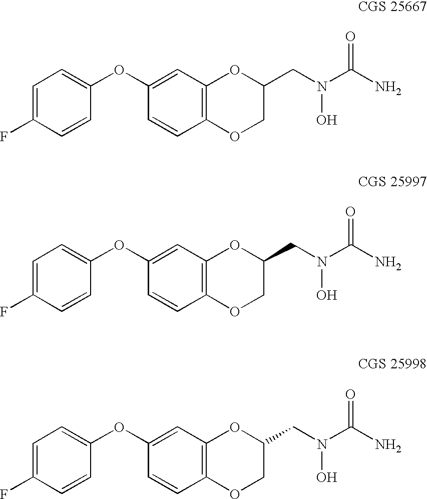

(29) CGS-23885, described ins for example, J. Med. Chem. 36, 3580 (1993).

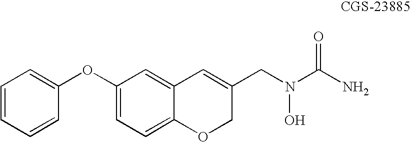

(30) CI-986

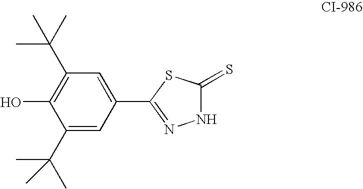

(31) CT 3 (synonyms: ajumelic acid, DMH-11C, HU 239), described in, for example, J. Med. Chem. 35, 3153 (1992).

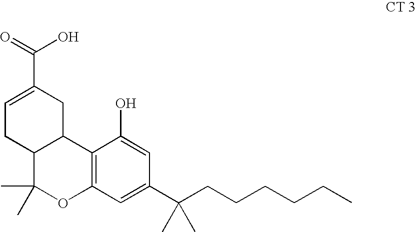

(32) CV 6504, described in, for example, Ann. Oncol. 11, 1165 (2000).

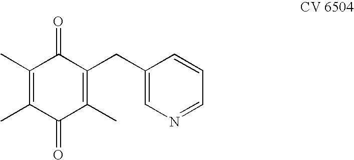

(33) Darbufelone (synonyms: CI-1004, PD 136095-0073) and analogues thereof, described in, for example, Arthritis and Rheumatism 42 (Suppl.) 404 (1999), ibid 42 (Suppl.) 81 (plus poster) (1999) and J. Med. Chem. 37, 322 (1994).

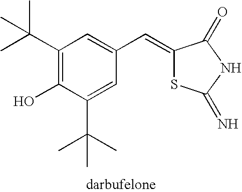

(34) Docebenone (synonym AA861) and analogues thereof, described in, for example, Int. Arch. Allergy and Immunol. 100, 178 (1993) and Biochim. Biopys. Acta 713, 470 (1982).

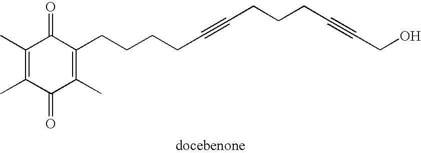

(35) DuP 654, described in, for example, J. Med. Chem. 33, 360 (1990).

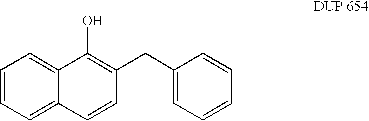

(36) XA 547, described in, for example, BTG International Inc. Company Communication 15 Oct. 1999, and Bioorg. Med. Chem. 3, 1255 (1995).
(37) E-3040

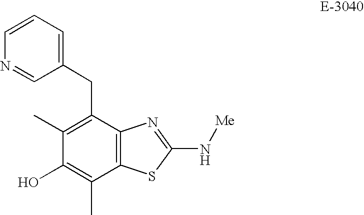

(38) E 6080, described in, for example, Res. Commun. Mol. Pathol. Pharmacol. 86, 75 (1994).

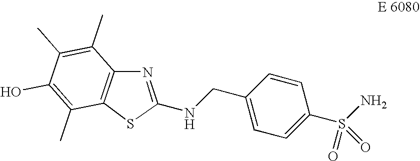

(39) E 6700.

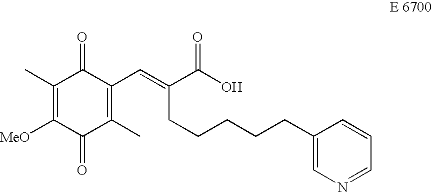

(40) Epocarbazolin A, a compound isolated from Streptomyces anulatus T688-8 and described in, for example, J. Antibiotics 46, 25 (1993).

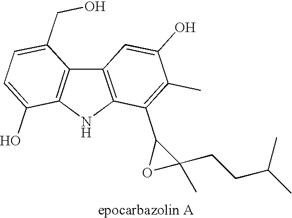

(41) ER 34122, described in, for example, Inflamm. Res. 47, 375 (1998).

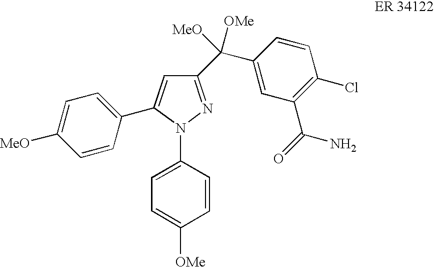

(42) ET 615, described in, for example, Exp. Dermatol. 2, 165 (1993).

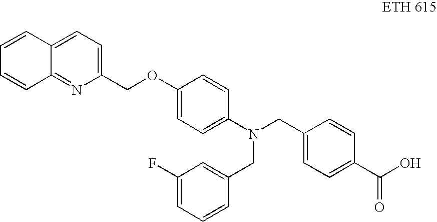

(43) F 1322, described in, for example, XV International Congress of Allergology and Clinical Immunology (Suppl 2) 325 (1994).

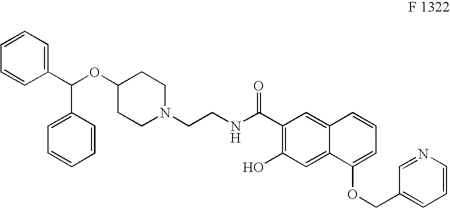

(44) Flezalastine (synonyms: D 18024, IDB 18024), described in, for example, Allergy (Suppl.) 47, 47 (1992).

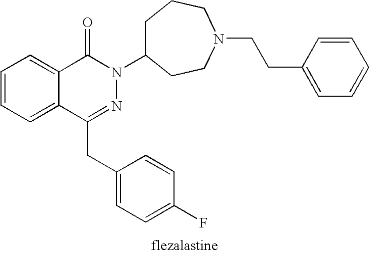

(45) Azelastine, described in, for example, Int. Arch. Allergy and Applied Immunol. 90, 285 (1989).

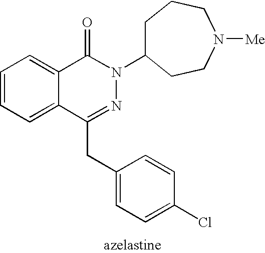

(46) FPL 62064, described in, for example, Agents and Actions 30, 432 (1990).

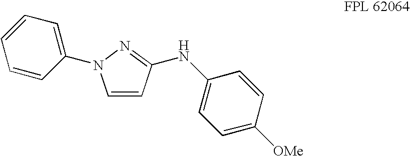

(47) FR 110302, described in, for example, Am. Rev. Resp. Dis. 145, A614 (1992).

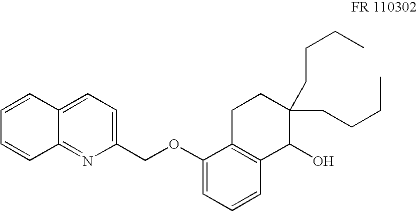

(48) HP 977 and P 10294, described in, for example, J. Med. Chem. 39, 246 (1996).

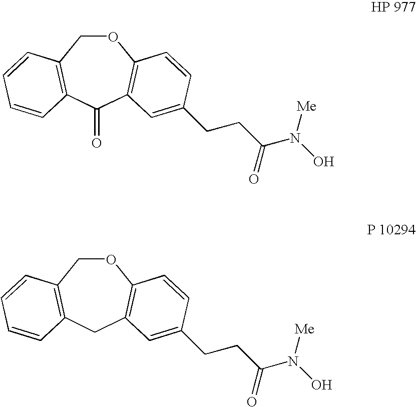

(49) P-8977

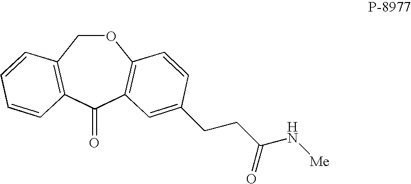

(50) HX-0835, described in, for example, Rinsho Iyaku. 11, 1577 & 1587 (1995).

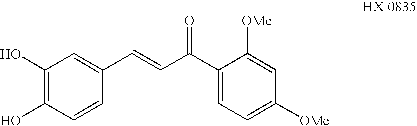

(51) HX-0836, described in, for example, J. Med. Chem. 36 3904 (1993).

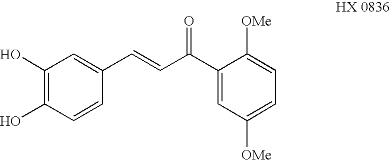

(52) The following compound, described in Bioorg. Med. Chem. Lett. 6, 93 (1996).

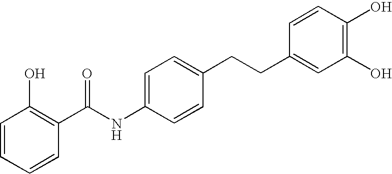

(53) Icodulinium (synonyms: CBS 113A, icoduline), described in, for example, Arzneimittel-Forschung (Drug Research) 39, 1242 & 1246 (1989).

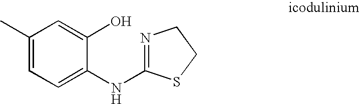

(54) KC-11404, described in, for example, Eur. Resp. J. 7 (Suppl. 18), 48 (1994).

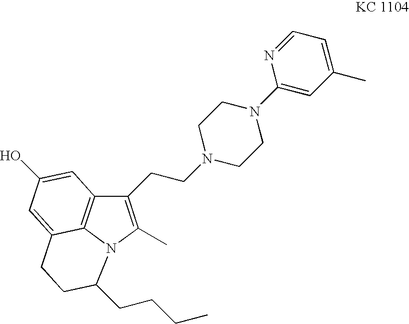

(55) KC-11425

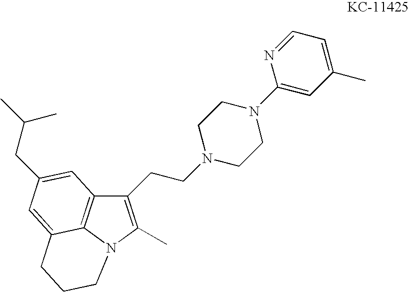

(56) KM 4.

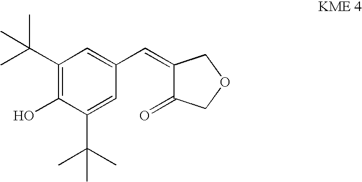

(57) L 651392, described in, for example, Adv. Prostaglandin, Thromboxane and Leukotriene Res. 17, 554 (1987).

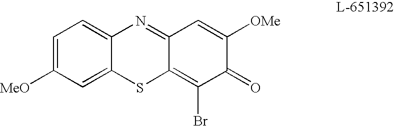

(58) L 651896.

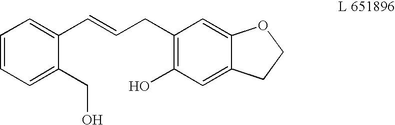

(59) L 6523.43.

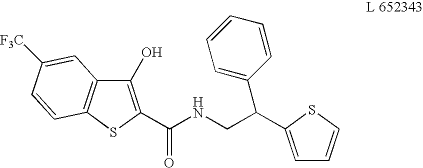

(60) L 653150.

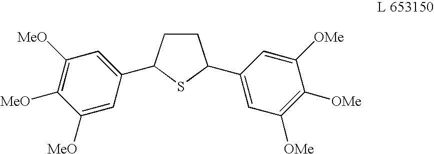

(61) L-656224, described in, for example, J. Gastroenterol. Hepatol. 11, 922 (1996).

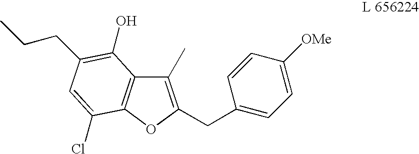

(62) L-702539, described in, for example, J. Med. Chem. 37, 512 (1994).

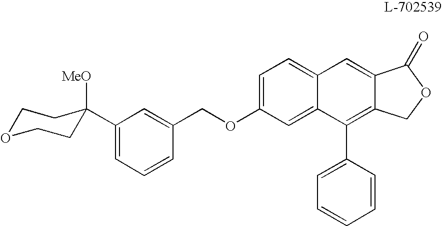

(63) L-670630.

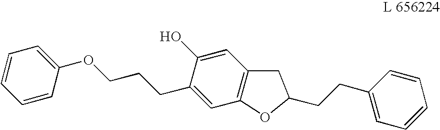

(64) L-691816, described in, for example, Curr. Opin. Invest. Drugs 2, 683 (1993).

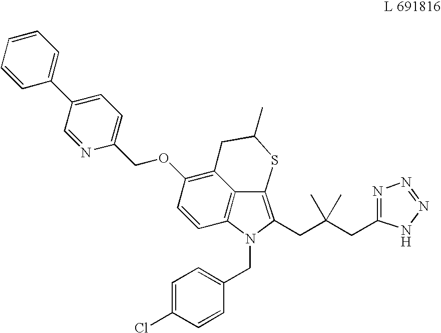

(65) L 699333, described in, for example, J. Med. Chem. 38, 4538 (1995).

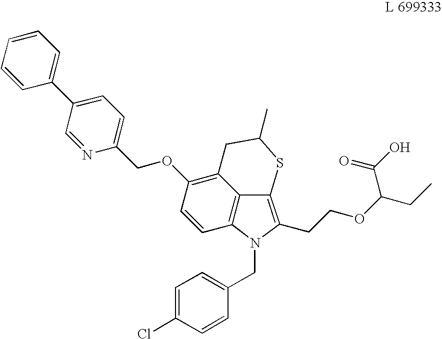

(66) L 739010.

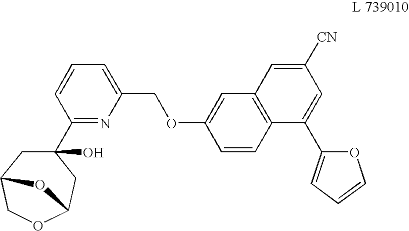

(67) Lagunamycin, described in, for example, J. Antibiotics 46, 900 (1993)

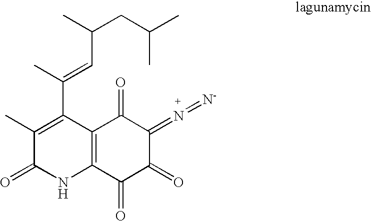

(68) Licofelone (synonym: ML 3000), described in, for example, Eur. J. Pharm. 453, 131 (2002) and J. Med. Chem. 37, 1894 (1994).

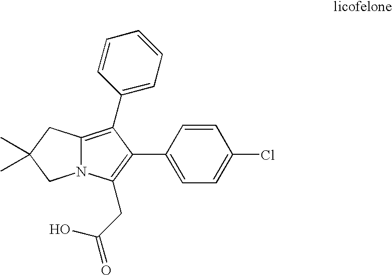

(69) PD 145246.

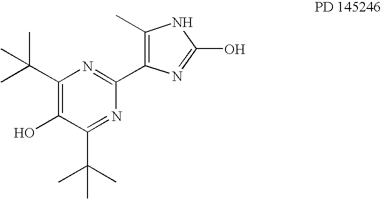

(70) R 840 (synonym: S 26431).

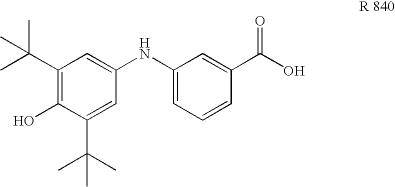

(71) R 68151, described in, for example, Arch. Dermatol. 128, 993 (1992).

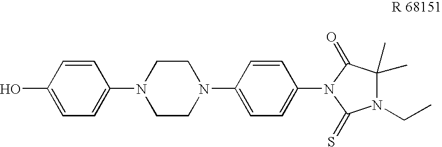

(72) R 85355, described in, for example, Skin Pharmacol. 9, 307 (1996).

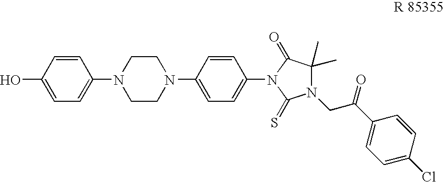

(73) REV 5901 (synonyms: PF 5901, Revlon 5901, RG 5901), described in, for example, J. Allergy Clin. Immunol. 91, 214 (1993).

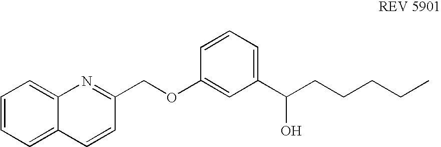

(74) RWJ 63556, described in, for example, 214th Am. Chem. Soc. Nat. Meeting. abstr. MEDI 091 (1997).

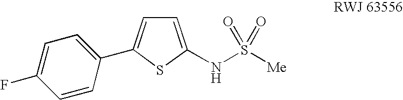

(75) S 19812, described in, for example, Mediators of Inflammation 8 (Suppl. 1), 134 & 135 (1999).

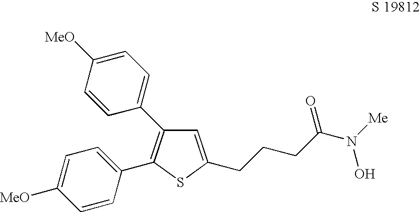

(76) SC 45662, described in, for example, J. Allergy and Clin. Immunol. 89, 208 (1992)

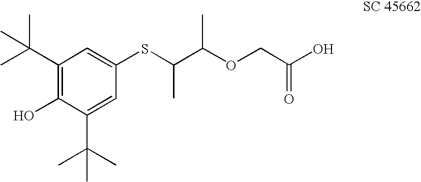

(77) SC-41661A

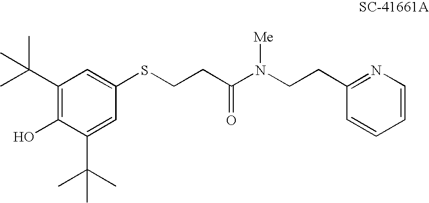

(78) SCH 40120.

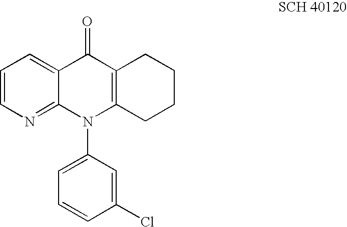

(79) SKF-86002

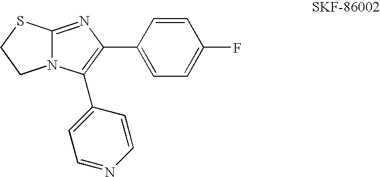

(80) SKF 104351 and SKF 105809.

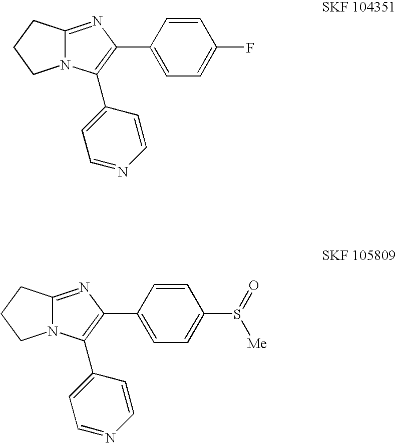

(81) SKF-107649, described in, for example, J. Med. Chem. 39, 5035 (1996).

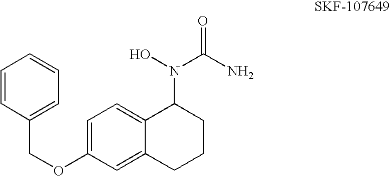

(82) T0757 and T0799), described in, for example, Jap. J. Pharmacol. 66, 363 (1994)

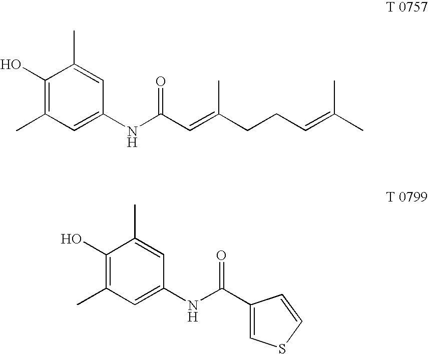

(83) TA 270, described in, for example, Naunyn-Schmiedeberg's Arch. Pharmacol. 358 (Suppl. 2) 737 (1998).

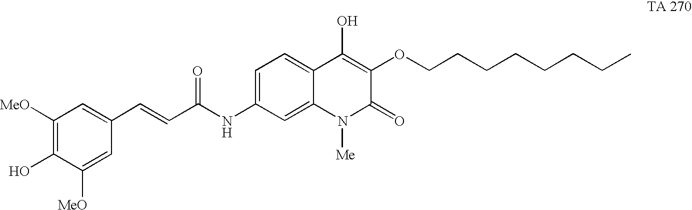

(84) Tagorizine (synonym: AL 3264), described in, for example, Jap. J. Pharmacol. 65, 19 (1994) and ibid 64 (Suppl. 1), 312 (1994)

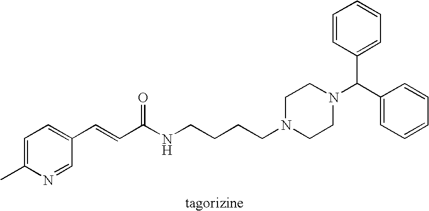

(85) Tepoxalin (synonyms: ORF 20485, RWJ 20485), described in, for example, J. Pharmacol. Exp. Therap. 271, 1399 (1994).

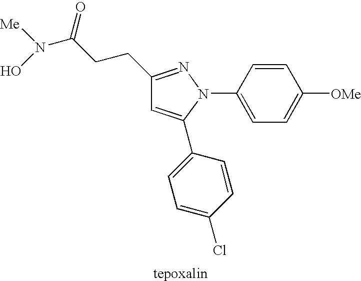

(86) UPA 780, described in, for example, Inflamm. Res. 44 (Suppl. 3), 273 (1995).

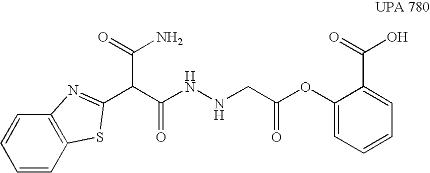

(87) VUFB 19363.

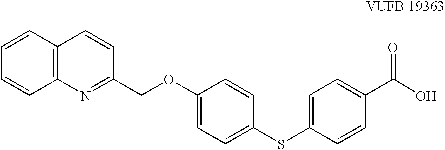

(88) VZ 564, described in, for example, Arzneimittel-Forschung (Drug Research) 25, 155 (1995).

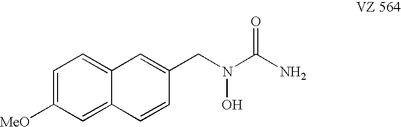

(89) The following compound, described in J. Med. Chem. 40, 819 (1997).

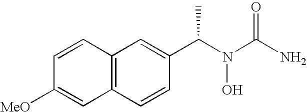

(90) WAY 120739.

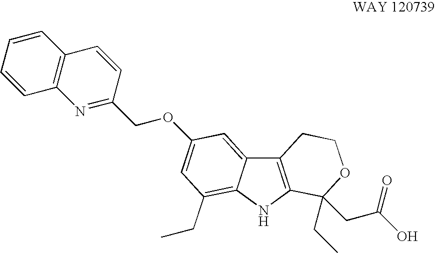

(91) WAY 121520, described in, for example, Agents and Actions 39 (Spec. issue C1) C30 (1993) and Exp. Opin. Invest. Drugs 6, 279 (1997).

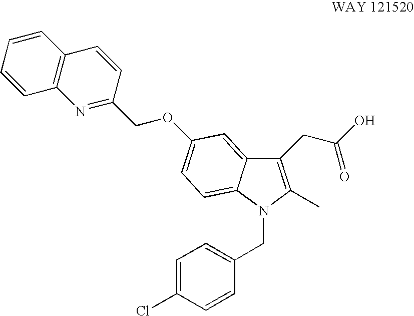

(92) WAY-126299A, described in, for example, Inflamm. Res. 44 (Suppl. 2), 170 (1995).

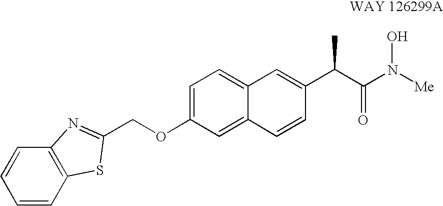

(93) WAY-125007, described in, for example, WO 04/004773

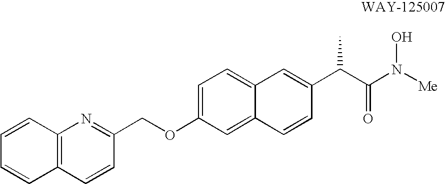

(94) WHIP 97, described in, for example, 216th Am. Chem. Soc. Nat. Meeting. abstr. MEDI 363 (1998).

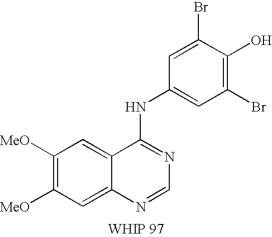

(95) WY 28342, described in, for example, J. Med. Chem. 38, 1473 (1995).

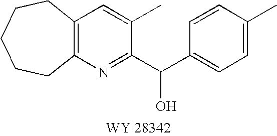

(96) WY 50295 (the S-enantiomer of WY 49232), described in, for example, Eur. J. Pharmacol. 236, 217 (1993).

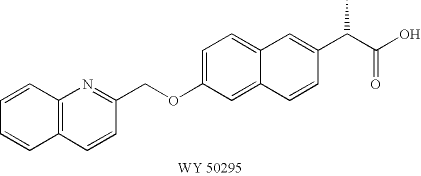

(97) D 2138 (synonym: ICI D 2138), described in, for example, Asthma 95: Theory to Treatment 15 (1995) and Trends in Pharm. Sci. 13, 323 (1992).

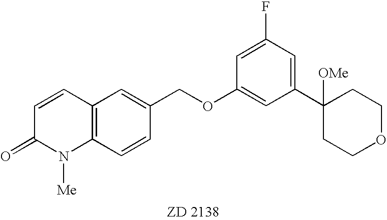

(98) ZM 230487 (synonym: ICI 230487), described in, for example, Inpharma 660, 9 (1994).

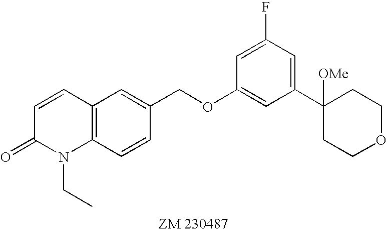

(99) ZD 4007 and ZD 4407, described in, for example, EP 0 623 614.

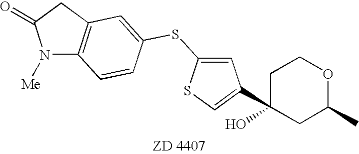

(100) ZD 7717, described in, for example, EP 0 462 813.

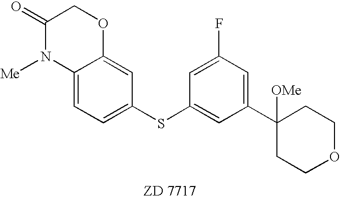

(101) ZM-216800.

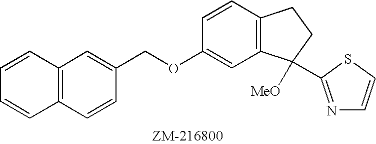

(102) CJ-12,918, and analogues thereof, described in, for example, Bioorg. Med. Chem. 11, 3879 (2003).

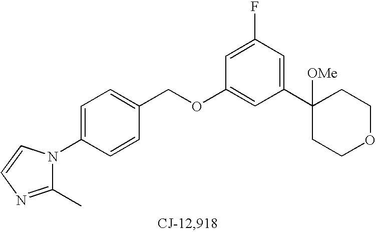

(103) Compounds described as mixed 5-LO/COX-2 inhibitors in Bioorg. Med. Chem. Lett. 12, 779 (2002), such as the following compound.

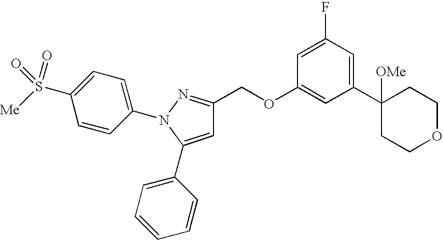

(104) AKBA (acetyl-11-keto-β-boswellic acid), described in, for example, Br. J. Pharmacol. 117, 615 (1996) and Eur. J. Biochem. 256, 364 (1998).

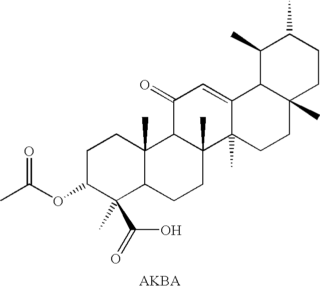

(105) Compounds described as dual 5-LO and COX inhibitors in Eur. J. Med. Chem. 22, 147 (1997) and Arzneimittel-Forschung (Drug Research) 35, 1260 (1985), such as 2-acetylthiophene-2-thiazolylhydrazone (CBS-1108) and N-phenylbenzamidrazone.

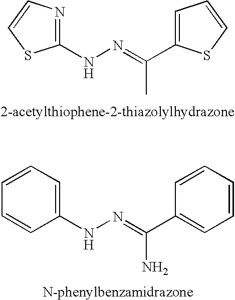

(106) Boswellin (an extract from Boswellia serrata), described in, for example, Fifth Chemical Congress of North America, Abstract 01/1351 (1997) and ibid. Abstract 01/1350 (1997).
(107) 2,4,6-triiodophenol, described as a 5-LO inhibitor in, for example, U.S. Pat. No. 5,985,937.
(108) Nicaraven, described in, for example, Curr. Opin. Invest. Drugs 4, 83 (2003).

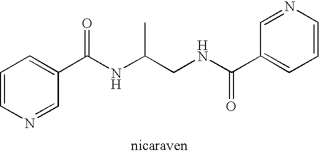

(109) Tenidap, described in, for example, EP 0 156 603, U.S. Pat. No. 4,556,672, Arthritis Rheum. 31, Suppl. S52 (1988) and P. Katz et al., ibid. S52.

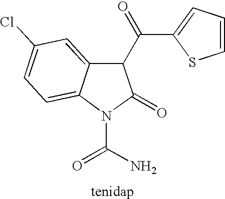

(110) Cyclic hydrazides described as 5-LO inhibitors in J. Med. Chem. 39, 3938 (1996), such as phenidone, 1-phenyl-2H-tetrahydropyridazin-3-one, and 1-phenylperhydro-1,2,4-tetrahydropyrdazin-3-one.

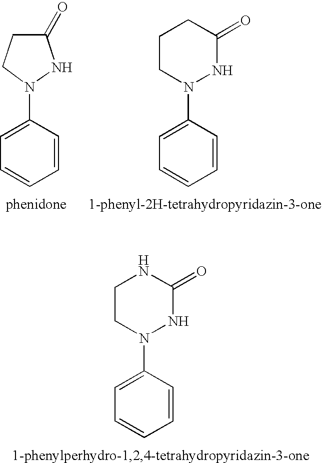

(111) ICI-207968, described in, for example, J. Med. Chem. 34, 1028 (1991).

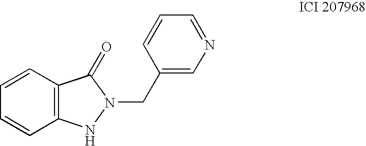

(112) ICI 211965, and other (methoxyalkyl)thiazoles, described in, for example, J. Med. Chem. 34, 2176 (1991).

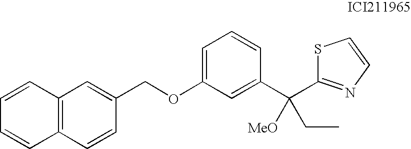

(113) 2,3-Dihydro-5-benzofuranols described in J. Med. Chem. 32, 1006 (1989), such as the following compound.

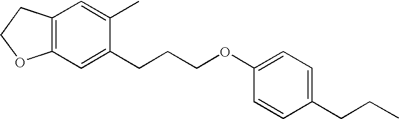

(114) 2,6-Di-tert-butylphenol derivatives described in Bioorg. Med. Chem. 11, 4207 (2003), such as tebufelone, R-830, and S2474.

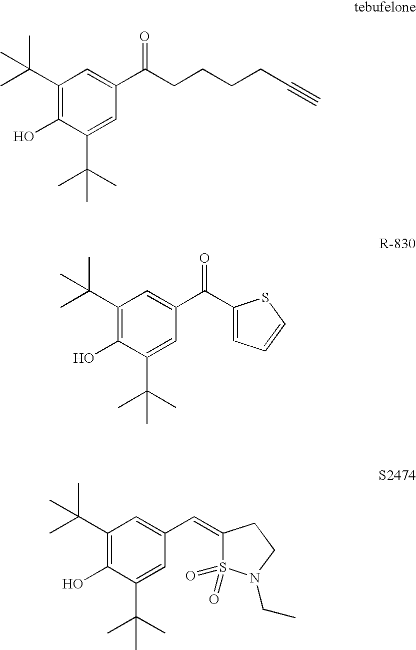

(115) 7-tert-Butyl-2,3-dihydro-3,3-dimethylbenzofurans described as 5-LO/COX-2 inhibitors in J. Med. Chem. 41, 1112 (1998), such as PGV-20229.

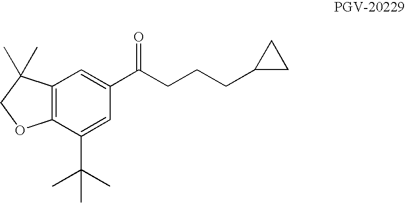

(116) Compounds described as dual 5-LO/COX inhibitors in Eur. J. Med. Chem. 35, 1897 (2003), such as the following compound.

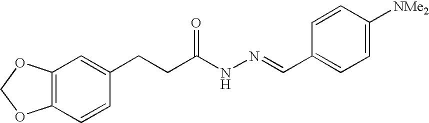

(117) Helenalin, a sesquiterpene lactone that can be isolated from several plant species of the Asteraceae family, described in, for example Biochem. Pharm. 62, 903 (2001).
(118) AS-35, (9-[(4-acetyl-3-hydroxy-2-n-propylphenoxy)methyl]-3-(1H-tetrazol-5-yl)-4H-pyrido[1,2-a]pyrimidinone), described in, for example, Int. J. Immunopharmacol. 22, 483 (2000).

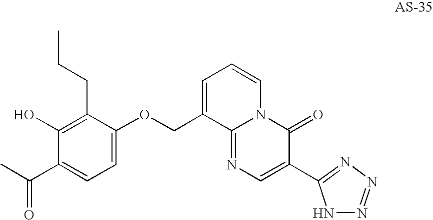

(119) Magnolol, described in, for example, Planta Medica 65, 222 (1999).

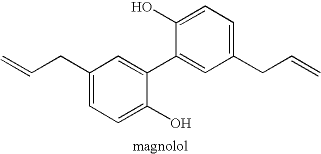

(120) Honokiol, extracted from Chinese herbal medicine, and described in, for example, Arch. Allergy and Immunol. 110, 278 (1996).

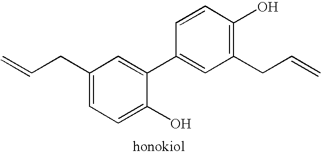

(121) Chrysarobin.

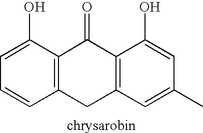

(122) E-3040.

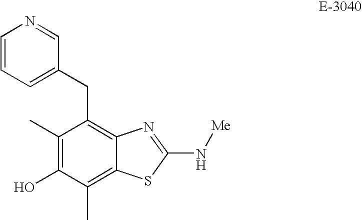

(123) Flobufen, described in, for example, Chirality 16, 1 (2004).

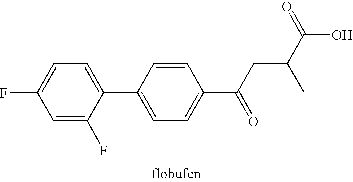

(124) YPE-01, described in, for example, Eur. J. Pharmacol. 404, 375 (2000).

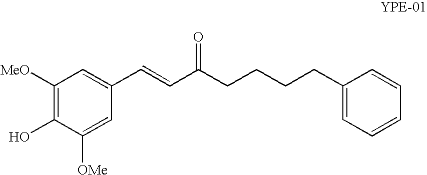

(125) BW-A137C

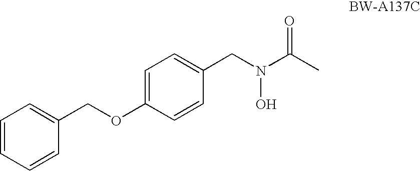

(126) LY-233569

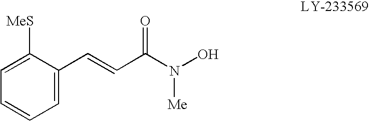

(127) PD-138387

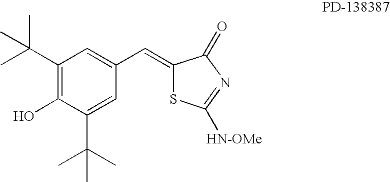

(128) SB-210661

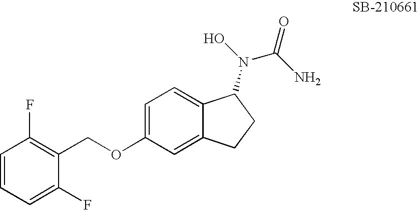

(129) DuP-983

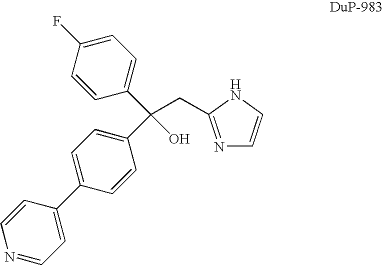

(130) BTS-71321

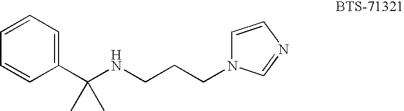

(131) Piripost, described in, for example, Toxicon. 24, 614 (1986).
(132) MK-866, described in, for example, Eur J Pharmacol 205, 259 (1991).
(133) UCB 62045, described in, for example, Chest 123, 371S (2003).
(134) ONO-LP-049, described in, for example, J. Immunol. 140, 2361 (1988).
(135) 3323W, L-697198, L-7080780, FR-122788, CMI-206, FPL-64170 and PD-089244
   and commonly employed salts thereof. Preferred 5-lipoxygenase inhibitors include zileuton or, more particularly, azelastine.

Other specific 5-LO inhibitors that may be mentioned include those described in the review articles Prog. Med. Chem., G. P. Ellis and D. K Luscombe, Elsevier 29, 1 (1992) and J. Med. Chem. 14, 2501 (1992).

Specific inhibitors of FLAP that may be mentioned include the following.
(a) L-674,573, and related FLAP inhibitors (e.g. L-655,238), described in, for example, Mol. Pharmacol. 40, 22 (1991).

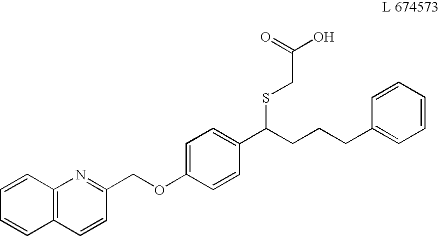

(b) L-674,636, described in, for example, J. Med. Chem. 38, 4538 (1995).

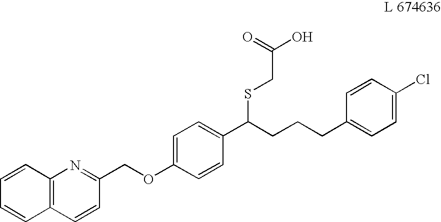

(c) L-689,037, and photoaffinity analogues [¹²⁵I]-669,083 and [¹²⁵I]-691,678, described in, for example, Mol. Pharmacol. 41, 267 (1992).

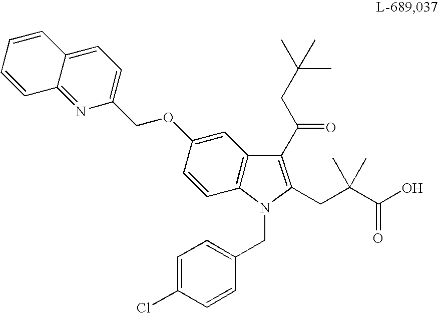

(d) L-705,302, described in, for example, J. Med. Chem. 38, 4538 (1995).

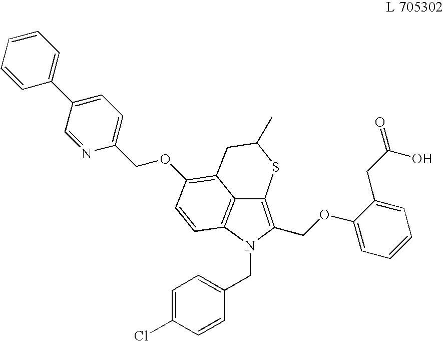

(e) MK-886 (synonyms: L663536, MK 0886), described in, for example, U.S. Pat. No. 5,081,138, Am. Rev. Resp. Dis. 147, 839 (1993), Eur. J. Pharmacol. 267, 275 (1994), The Search for Anti-Inflammatory Drug. 233 (1995) Eds.:V. J. Merluzzi and J. Adams, Boston, Birkhäuser.

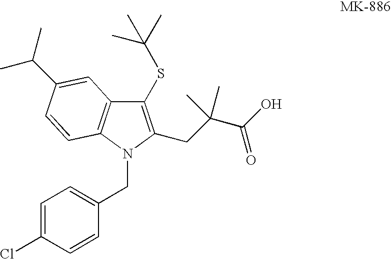

(f) Compounds structurally related to MK-886, described in, for example, WO 93/16069, U.S. Pat. No. 5,308,850 and WO 94/13293
(g) Quiflapon (synonyms: MK-591, L 686708), described in, for example, J. Physiol. Pharmacol. 70, 799 (1992) and J. Lipid Mediators 6, 239 (1993).

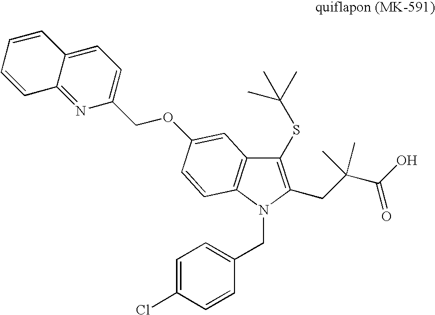

(h) BAY X 1005, described in, for example, Thorax 52, 342 (1997).

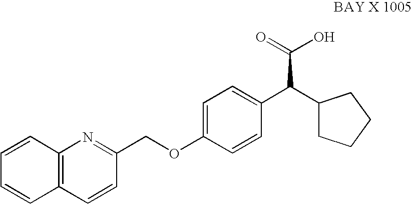

(i) BAY Y 105, described in, for example, Arthritis and Rheumatism 39, 515 (1996) and Drug & Market Devel. 7, 177 (1996).

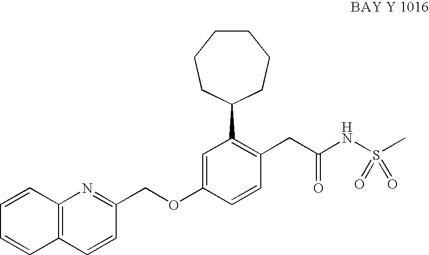

(j) VML 530 (synonym: ABT 080), described in, for example, Pharmacologist 39, 33 (1997)

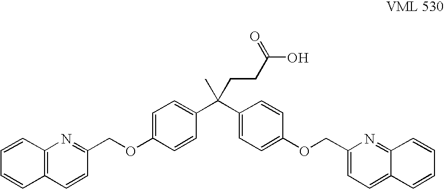

and commonly employed salts thereof.

Inhibitors of LTA₄ hydrolase that may be mentioned include the following.
(A) Compounds described as LTA₄ hydrolase inhibitors in U.S. Pat. No. 5,455,271 and WO 94/00420, for example:

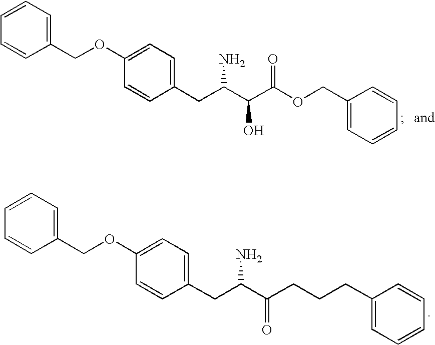

(B) Compounds described as LTA₄ hydrolase inhibitors in J. Med. Chem. 36, 211 (1993) and J. Am. Chem. Soc. 114, 6552 (1992), such as the following compound

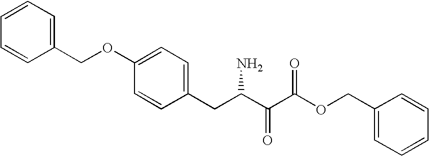

(C) Compounds identifiable by the method of claim 24 of WO 00/50577.
(D) Compounds described as LTA₄ hydrolase inhibitors in U.S. Pat. No. 6,506,876, such as SC-56938.

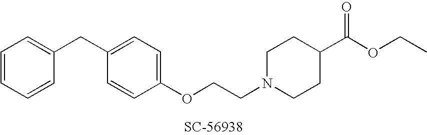

(E) Analogues of SC-56938, described in, for example, Bioorg. Med. Chem. Lett. 12, 3383 (2002).
(F) Compounds described as LTA₄ hydrolase inhibitors in U.S. Pat. No. 5,719,306, for example:

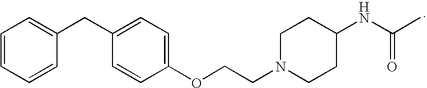

(G) Compounds described as LTA₄ hydrolase inhibitors in WO 96/11192, such as:

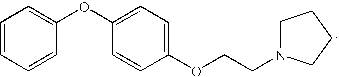

(H) Compounds described as LTA₄ hydrolase inhibitors in U.S. Pat. No. 6,265,433 and WO 98/40364, for example:

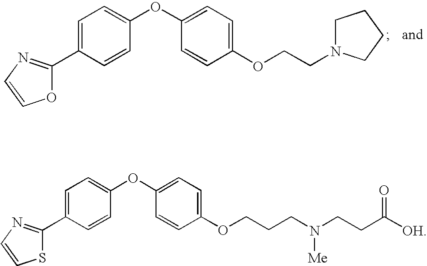

(I) Compounds described as LTA₄ hydrolase inhibitors in U.S. Pat. No. 6,506,876 and WO 96/10999, such as:

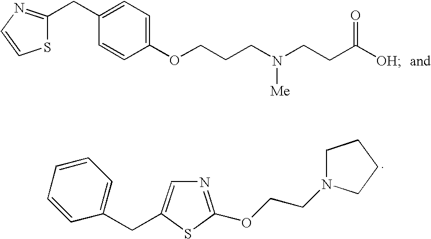

(J) Compounds described as LTA₄ hydrolase inhibitors in WO 98/40370, such as:

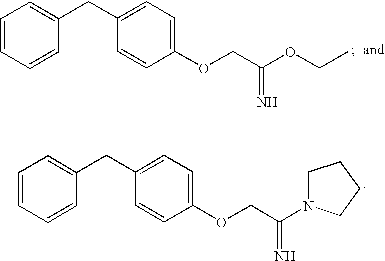

(K) Compounds described as LTA₄ hydrolase inhibitors in WO 99/40354.
(L) Compounds (3-oxiranylbenzoic acids) described as LTA₄ hydrolase inhibitors in EP 0 360 246, such as:

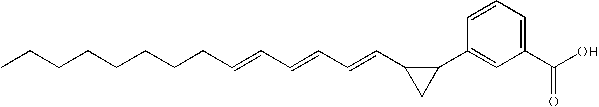

(M) 20,20,20-Trifluoroleukotriene B4 derivatives, described in, for example, JP 01211549 A2, such as the following compound.

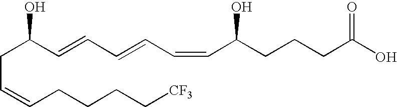

N) Compounds described as LTA₄ hydrolase inhibitors in EP 1 165 491 and WO 00/059864, such as 2-amino-6-(4-benzylphenoxy)hexanoic acid:

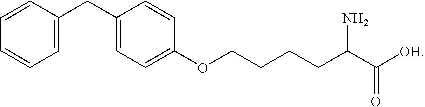

(O) Compounds described as LTA₄ hydrolase inhibitors in U.S. Pat. No. 6,436,973 and WO 00/017133, such as (2S,3R)-2-amino-3-(benzyloxy)butane-1-thiol:

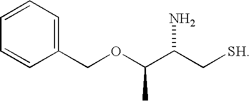

(P) Compounds described as LTA₄ hydrolase inhibitors in Bioorg. Med. Chem. 3, 969 (1995), such as:

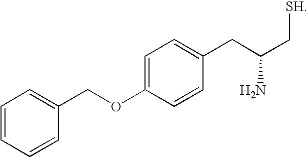

(Q) [4-(ω-Arylalkyl)phenyl]alkanoic acids described as LTA₄ hydrolase inhibitors in DE 4121849 A1, such as:

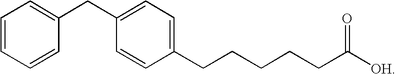

(R) Aralkthienylalnoates described as LTA₄ hydrolase inhibitors in DE 4118173 A1, such as:

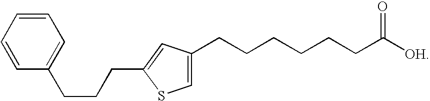

(S) ω-[(4-Arylalkl)thien-2-yl]alkanoates described as LTA₄ hydrolase inhibitors. in DE 4118014 A1, such as:

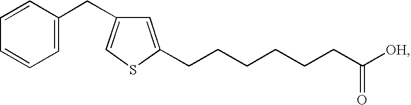

(T) Compounds described as LTA₄ hydrolase inhibitors in J. Med. Chem. 35, 3156 (1992), such as RP64966:

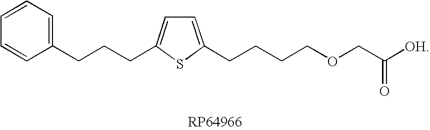

(U) Compounds structurally related to RP66153 and described in J. Med. Chem. 35, 3170 (1992).
(V) 2-Hydroxyphenyl-substituted isoxazoles described as LTA₄ hydrolase inhibitors in DE 431-4966 A1, such as:

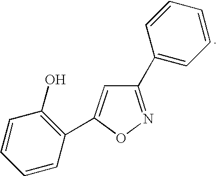

(W) Bestatin, described in, for example, J. Nat. Cancer Institute 95, 1053 (2003).

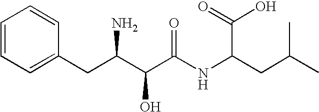

(X) SC-22716 (1-[2-(4-phenylphenoxy)ethyl]pyrrolidine), described in, for example, J. Med. Chem. 43, 721 (2000).

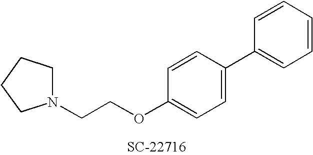

(Y) SC57461A, described in, for example, J. Med. Chem. 45, 3482 (2002) and Curr. Pharm Design 7, 163 (2001).

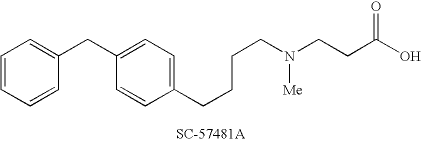

(Z) Imidazopyridines and purines described as LTA₄ hydrolase inhibitors in Bioorg. Med. Chem. Lett. 13, 1137 (2003).
(AA) Captopril, described in, for example, FASEB Journal 16, 1648 (2002).

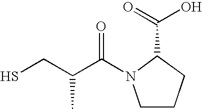

(AB) Hydroxamic acid derivatives described as LTA₄ hydrolase inhibitors in WO 99/40910, such as:

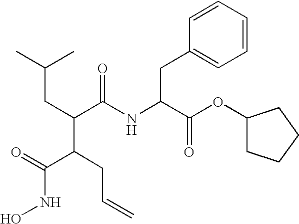

(AC) AB5366, described in, for example, JP 11049675 A2.
(AD) SA6541, described in, for example, WO 96/27585, Life Sci. 64, PL51-PL56 (1998) and Eur. J. Pharmacol. 346, 81 (1998).

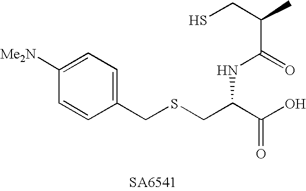

(AE) Compounds containing N-mercaptoacylprolines described as LTA₄ hydrolase inhibitors in JP 10265456 A2, such as:

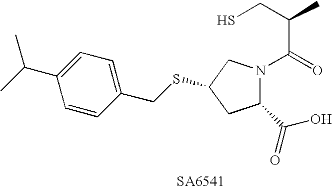

(AF) Amphotericin B, described in, for example, Prostaglandins, Leukotrienes and Essential Fatty Acids 58, 105 (1998).

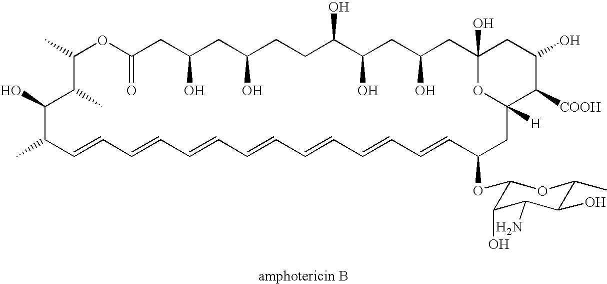

(AG) 14,15-Dehydroleukotriene A4, described in, for example, Biochem. J. 328, 225 (1997).
(AH) 8(S)-amino-2(R)-methyl-7-oxononanoic acid, produced by Streptomyces diastaticus and described in, for example, J. Natural Products 59, 962 (1996).

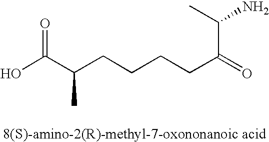

(AI) The hydroxamic acid-containing. peptide kelatorphan, described in, for example, Bioorg. Med. Chem. Lett. 5, 2517 (1995).
(AJ) Amino hydroxamic acids described as inhibitors of LTA₄ hydrolase in Bioorg. Med. Chem. 3, 1405 (1995), such as:

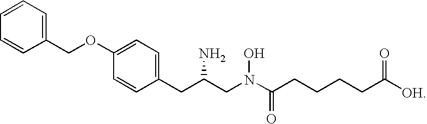

(AK) α-Keto-β-amio esters and thioamines described as inhibitors of LTA₄ hydrolase in J. Pharmacol. Exp. Therap. 275, 31 (1995).
(AL) N-(phenylbutanoyl)leucines described as inhibitors of LTA₄ hydrolase in JP 05310668 A2

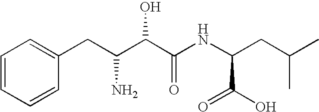

and commonly employed salts thereof.

Other specific inhibitors of LTA₄ hydrolase that may be mentioned include those described in the review articles Curr. Pharm. Design 7, 163 (2001) and Curr. Med. Chem. 4, 67 (1997).

Antagonists of LTB₄ receptors (e.g. BLT1) that may be mentioned include the following.
(i) Compounds described as LTB₄ receptor antagonists in U.S. Pat. No. 6,291,530, such as (E)-[5-(2-diethylcarbamoyl-1-methylvinyl)-2-(2,6-difluoro-benzyloxy)phenoxy]acetic acid:

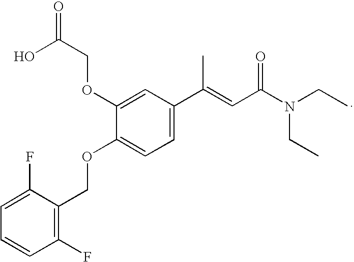

(ii) Compounds described as LTB₄ receptor antagonists in US 2002/0128315, such as 4-(4-phenylpiperidinylmethyl)benzoic acid 4-amidinophenyl ester and 4-(2-phenylimidazolylmethyl)benzoic acid 4-amidinophenyl ester:

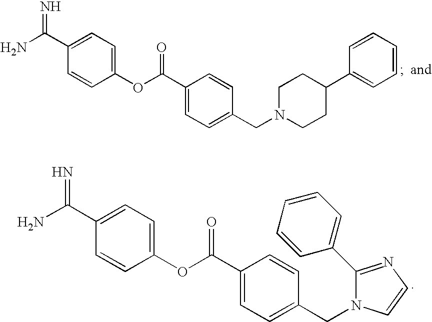

(ii) Compounds described as LTB₄ receptor antagonists in US 2004/0053962, such as 2-(2-propyl-3-(3-(2-ethyl-4-(4-fluorophenyl)-5-hydroxyphenoxy)propoxy)phenoxy)benzoic acid:

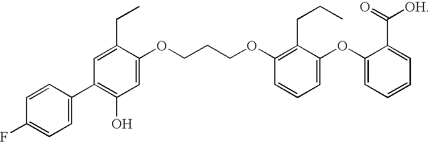

(iv) BIIL, described in, for example, J. Pharmacol. Exp. Therap. 297, 458 (2001) and WO 02/055065.

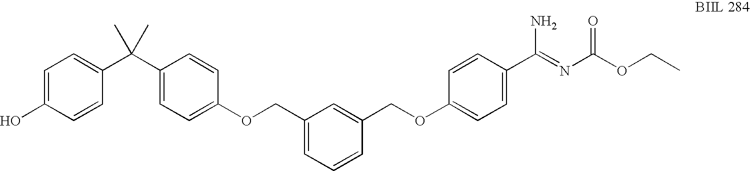

(v) CP 105696 and CP 195543, described in, for example, J. Pharmacol. Exp. Therap. 285, 946 (1998).

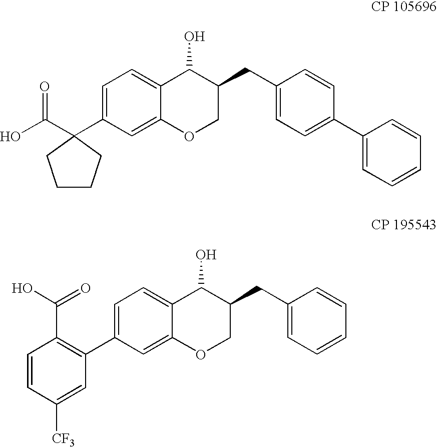

(vi) LY 210073

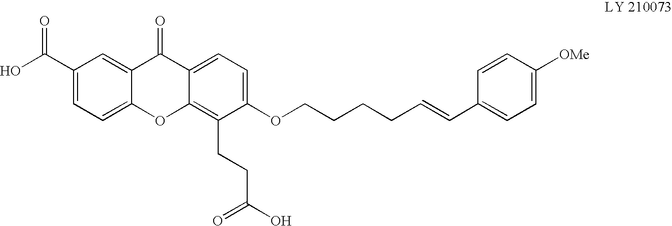

(vii) LY 223982 (synonyms: CGS 23131, SK 107324).

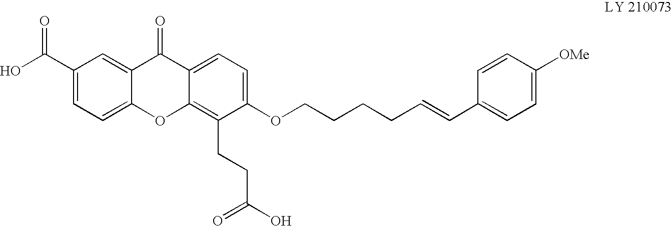

(viii) LY 255283 (synonyms: CGS 23356, LY 177455), described in, for example, Eur. J. Pharmacol. 223, 57 (1992).

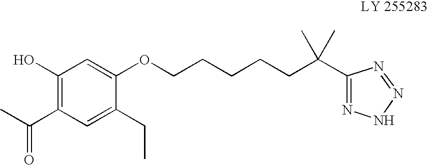

(ix) LY 292728.

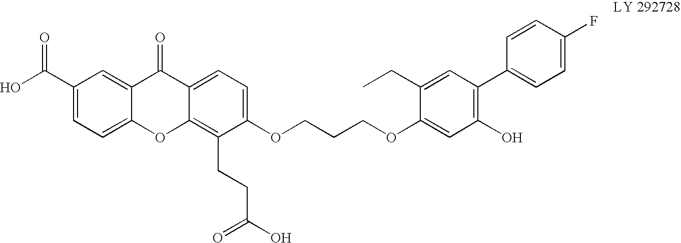

(x) LY 293111 (synonym: VML 295), described in, for example, Drugs of the Future 21, 610 (1996), Clin Cancer Res. 8, 3232 (2002) and WO 01/085166.

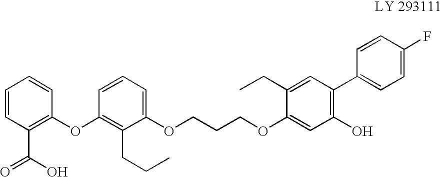

(xi) LTB 019.
(xii) Moxilubant (synonym: CGS 25019C), described in, for example, Exp. Opin. Therap. Patents 5, 127 (1995).

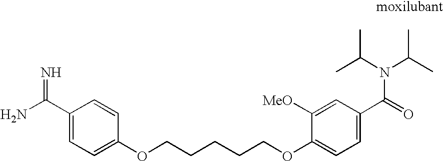

(xiii) Olopatidine (synonyms: allelock, ALO 4943A, KW 4679, Patanol®), described in, for example, Drugs of the Future 18, 794 (1993).

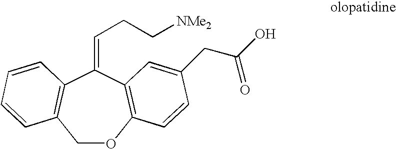

(xiv) ONO 4057 (synonym: LB 457), described in, for example, Gastroenterology 110 (Suppl.), 110 (1996).

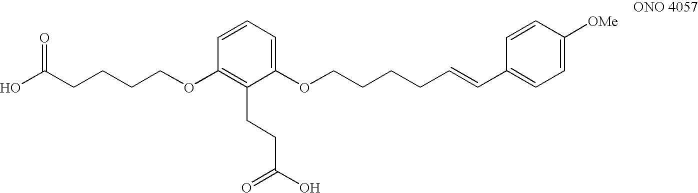

(xv) Ontazolast (synonym: BIRM 270), described in, for example, J. Pharm. Exp. Therap. 271, 1418 (1994).

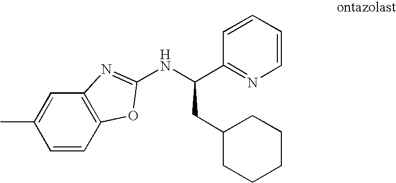

(xvi) PF 10042, described in, for example, Eur. J. Phamacol.—Enviromental Toxicology and Pharmacology Section 293, 369 (1995).

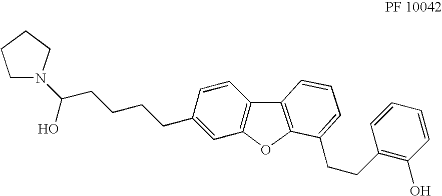

(xvii) RG 14893, described in, for example, Pharmacologist 34, 205 (1992).

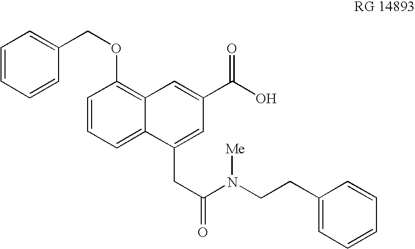

(xviii) RO 254094, described in, for example, ISSX Proceedings 6, 232 (1994).

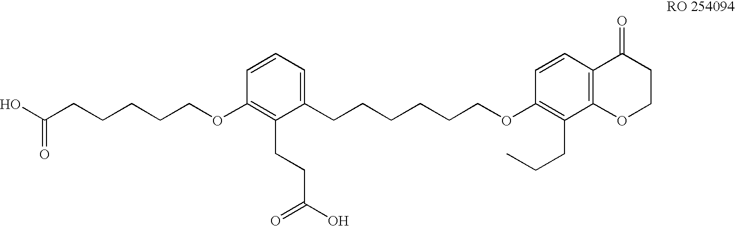

(xix) RP 66153.

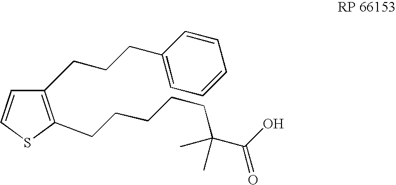

(xx) RP 66364.

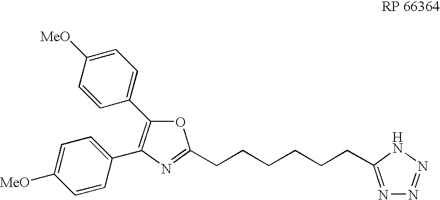

(xxi) RP 69698.

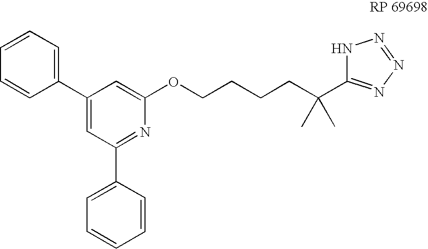

(xxii) SB 201146, described in, for example, Thorax 53, 137 (1998).

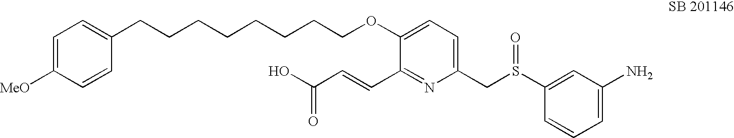

(xxiii) SB 201993, described in, for example, J. Med. Chem. 36, 2703 (1993).

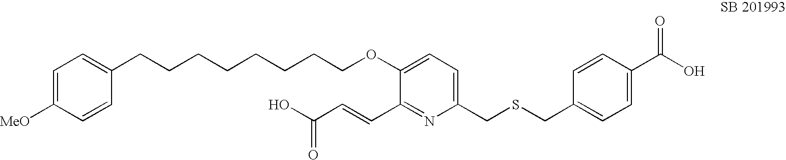

(xxiv) SC 41930, described in, for example, J. Pharmacol. Exp. Therap. 269, 917 (1994)

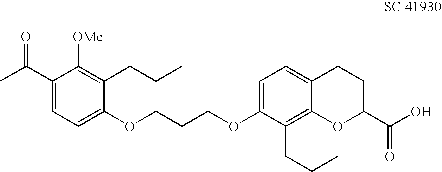

(xxv) SC 50605.

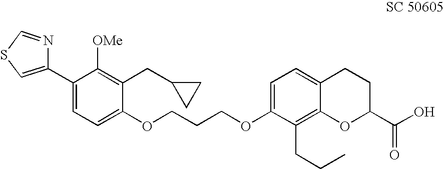

(xxvi) SC 51146.

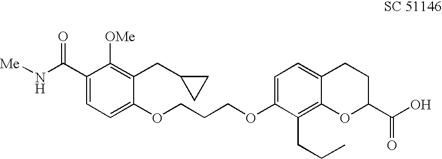

(xxvii) SC 53228, described in, for example, Inflammation Res. 44 (Suppl. 2), 143 (1995).

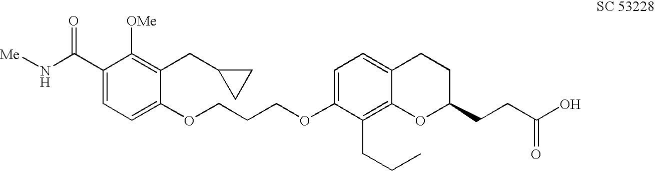

(xxvii) Ticolubant (synonym: SB 209247), described in, for example, Adv. Prostaglandin Thromboxane and Leukotriene Res. 23, 275 (1995).

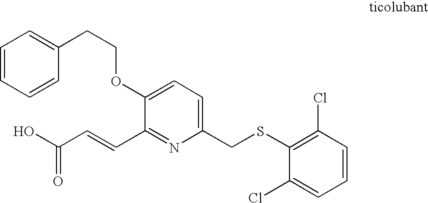

(xxix) U 75302 (synonyms: U 75485, U 77692, U 78489), described in, for example, Adv. Prostaglandin Thromboxane and Leukotriene Res. 23, 275 (1995).

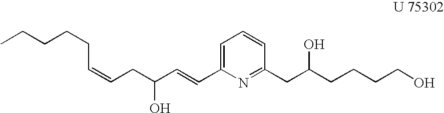

(xxx) VM 301 (synonyms: OAS 1000, pseudopterosin A methyl ether), described in, for example, Inflammation Res. 44, (Suppl. 3) 268 (1995).
(xxxi) ZD 158252, described in, for example, Inpharma 1094, 9 (1997).
(xxxii) ZK 158252, described in for example, Inpharma 1094, 9 (1997).

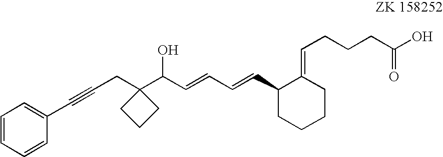

(xxxiii) U-75509, described in, for example, Am. J. Physiol. Heart Circ. Physiol. 2004, March 11 [Epub ahead of print].
(xxiv) CP-105,696, described in, for example, Br. J. Pharmacol. 139, 388 (2003).
(xxi) LY293111, described in, for example, Clin. Cancer Res. 8, 3232 (2002) and commonly employed salts thereof.

H₁ histamine receptor antagonists that may be mentioned include acrivastine, alimemazine, anatazoline, astemizole, azatadine, azelastine, bamipine, bepotasine, bromazine, bromopheniramine, buclizine, carbinoxamine, chlorocyclizine, chloropyramine, chlorophenamine, cinnarizine, clemastine, clemizole, clocinizine, cyclizine, cyproheptadine, deptropine, desloratdine, dexchlorpheniramine, dimenhydrinate, dimetindene, dimetotiazine, diphenhydramine, piphenylpyraline, doxylamine, ebastine, embramine, emedastine, epinastine, fexofenadine, flunarizine, homochlorocyclizine, hydroxyzine, isothipendyl, levocarbastine, loratidine, mebhydroline, meclozine, mepyramine, mequitazine, methdilazine, mizolastine, niaprazine, olopatadine, oxatomide, oxomemazine, phenindamine, pheniramine, phenyltoloxamine, pimethixene, pipinhydrinate, promethazine, propiomazine, quifenadine, ruputadine, setastine, terfenadine, thenyldiamine, thiethylperazine, thonzylamine, tolpropamine, trimethobenzamine, tripelennamine, triprolidine and tritoqualine and commonly employed salts thereof.

Active ingredients may be employed in combination.

Any pharmaceutically-acceptable salt of an antiinflammatory and/or antihistaminic active ingredient, as well as the free base form thereof may be used in the manufacture of compositions of the invention. Preferred salts include acetate salts, acetonate salts, aluminium salts, ammonium salts, arginine salts, bromide salts, butyrate salts, calcium salts, chloride salts, choline salts, citrate salts, diethanolamine salts, diethylamine salts, dipropionate salts, embonate salts, ethanolamine salts, ethylenediamine salts, formate salts, fumarate salts, fuorate salts, hydrobromide salts, hydrochloride salts, imidazole salts, lactate salts, lysine salts, magnesium salts, malate salts, maleate salts, malonate salts, meglumine salts, mesilate salts, morpholine salts, nitrate salts, phosphate salts, piperazine salts, potassium salts, propionate salts, sodium salts, succinate salts, sulfate salts, tartrate salts, teoclate salts, para-toluenesulfate salts, triethanolamine salts, triethylamine salts, valerate salts, etc and/or as described in “Handbook of Pharmaceutical Salt”, Eds. Stahl and Wermuth, Wiley, 2002, Chapter 12.

The amount of an antiinflammatory and/or anthistaminic active ingredient, or salt thereof that may be employed in preparation of compositions of the invention may be determined by the physician, or the skilled person, in relation to what will be most suitable for an individual patient. This is likely to vary with the nature of the active ingredient employed, the severity of the condition that is to be treated, as well as the species, age, weight, sex, renal function, hepatic function and response of the particular patient to be treated. It is preferred however that the compositions of the invention comprise all antiinflammatory and/or antihistaminic drug, or a salt thereof in an amount of from about 0.1 mg/mL to about 200 mg/mL calculated on the free-base form.

The total amount of active ingredient that may be present may be sufficient to provide a daily dose of drug per unit dosage that is appropriate for the active ingredient(s) that is/are employed. For example, this may be in the range about 20 μg to about 200 mg. The skilled person will appreciate that compositions of the invention may be dosed once or more times daily in one or more administrations in order to provide the aforementioned daily dose. Preferred ranges include from about 0.1 mg/mL to about 100 (e.g. about 70) mg/mL and, more particularly from about 0.2 mg/mL to about 50 mg/mL.

The above-mentioned dosages are exemplary of the average case; there can, of course, be individual instances where higher or lower dosage ranges are merited, and such are within the scope of this invention.

The term “liposome” will be well understood by those skilled in the art to include a structure consisting of one or more concentric spheres of polar lipid bilayers separated by water or aqueous buffer compartments.

Liposomes may be prepared by various methods using solvents, reduced pressure, two-phase systems, freeze drying, sonication etc. described, for instance, in Liposome Drug Delivery Systems, Betageri G V et al., Technomic Publishing AG, Basel, Switzerland, 1993, the relevant disclosures in which document are hereby incorporated by reference.

The term “polar lipid” will be well understood by the skilled person to include any lipid with a polar head-group and two fatty acid residues, which is capable of forming liposomes.

Polar lipids, such as those described hereinafter, may be of a natural and/or a synthetic/semi-synthetic origin. Mixtures of natural and synthetic/semi-synthetic polar lipids may also be employed in compositions of the invention.

Polar lipids that may be employed in compositions of the invention may thus be based on, for example, phospholipids, and in particular phosphatidylcholine (PC), phosphatidylglycerol (PG), phosphatidylinositol (PI), phosphatidic acid (PA), phosphatidylserine (PS), or mixtures thereof.

Phospholipids that may be employed in compositions of the invention comprise polar and non-polar groups linked to a backbone entity carrying hydroxyl groups, such as glycerol.

Phospholipids may also be represented by the general formula I

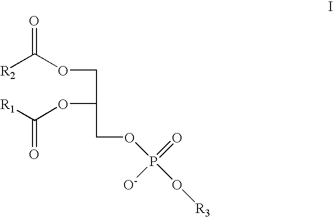

wherein R₁ and R₂ independently represent a saturated or unsaturated (e.g. alkenyl), branched or straight chain alkyl group having between 7 and 23 carbon atoms, preferably between 11 and 19 carbon atoms; and R₃ represents an amide or ester bonding group, such as
—CH₂—CH(OH)—CH₂OH (phosphatidylglycerol),
—CH₂—CH₂—N(CH₃)₃ (phosphatidylcholine),
—CH₂—CH₂—NH₂ (phosphatidylehanolamine),
—H (phosphatidic acid), or
—CH₂—CH(NH₂)—COOH (phosphatidylserine).

The phospholipid may be of natural origin. Natural phospholipids are preferably membrane lipids derived from various sources of both vegetable (e.g. rapeseed, sunflower, etc., or, preferably, soybean) and animal origin (e.g. egg yolk, bovine milk, etc.). Phospholipids from soybean, a major source of vegetable phospholipids, are normally obtained from the by-products (i.e. lecithins) in the refining of crude soybean oil by the degumming process. The lecithins are further processed and purified using other physical unit operations, such as fractionation and/or chromatography. Other phospholipids may be obtained, for example, by pressing various suitable seeds and grains, followed by solvent extraction and then further processing as described above. Phospholipids of natural origin that may be mentioned include for example those that are available under the tradenames Lipoid S75, Lipoid S100 and Lipoid S75-3N (Lipoid GmbH, Germany), which are all blends of several different phospholipids that are found in soybean.

The phospholipid may alternatively be of synthetic or semi-synthetic origin (i.e. prepared by chemical synthesis). For example, a multi-step chemical synthetic approach may be used in order to obtain the key phospholipid intermediates, 1,2-diacylglycerol, from (S)-1,2-isopropylideneglycerol, the latter providing the glycerol backbone that is characteristic of phospholipids. 1,2-Diacetylated phospholipids may then be obtained when the corresponding polar head group is attached via chemical synthesis to the 1,2-diacylglycerol intermediate. Generally, however, the origin of glycerol and the fatty acids used in the various steps may be of both natural and synthetic origin. Synthetic and/or semi-synthetic phospholipids that may be mentioned include dilaurylphosphatidylcholine (DLPC), dimyristolphosphatidylcholine (DMPC), dipalmitoylphosphatidylcholine (DPPC), dilaurylphosphatidylglycerol (DLPG), dimyristolphosphatidylglycerol (DMPG), dioleoylphosphatidylcholine (DOPC) and dioleoylphosphatidylglycerol (DOPG).

The polar lipid may alternatively comprise or, more preferably, consist of a glycolipid. In the context of the present invention, the term “glycolipid” designates a compound containing one or more monosaccharide residues bound by a glycosidic linkage to a hydrophobic moiety such as an acylglycerol, a sphingoid or a ceramide (N-acylsphigoid).

A glycolipid may be a glycoglycerolipid. In the context of the present invention, the term “glycoglycerolipid” designates a glycolipid containing one or more glycerol residues. According to a preferred aspect of the invention, the glycoglycerolipid comprises, or consists of, galactoglycerolipid, more preferably a digalactosyldiacylglycerol of the general formula II,

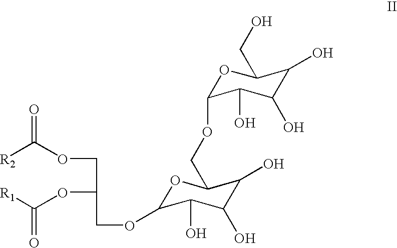

wherein R₁ and R₂ are as hereinbefore defined.

The glycolipid may alternatively be a glycosphingolipid. In the context of the present invention, the term “glycosphingolipid” designates a lipid containing at least one monosaccharide residue and either a sphingoid or a ceramide. The term may thus comprise neutral glycophigolipids, such as mono- and oligoglycosylsphingoids as well as oligo- and, more preferably, monoglycosylceramides. The term additionally comprises acidic glycosphingolipids such as sialoglycosphingolipids, uronoglycosphingolipids, sulfoglycosphingolipids, phosphoglycosphingolipids, and phosphonoglycosphingolipids. The glycosphingolipid can be ceramide, monohexosylceramide, dihexosylceramide, sphingomyelin, lysosphingomyelin, sphingosine, or a mixture thereof. Preferably the glycosphingolipid is sphingomyelin or products derived therefrom. The sphingomyelin content is preferably established by chromatographic methods. Sphingomyelin may be extracted from milk, preferably bovine milk, brain, egg yolk or erythrocytes from animal blood, preferably sheep. For the avoidance of doubt, synthetic and semi-synthetic sphingolipids are comprised by the invention.

The glycolipid may alternatively be a glycopbosphatidylinositol. In the context of the present invention, the term “glycophosphatidylinositol” designates a glycolipid containing saccharides glycosidically linked to the inositol moiety of phosphatidylinositols.

Preferred glycolipids include digalactosyldiacylglycerol (DGDG).

It is preferred that the polar lipid is based on a phospholipid and, more particularly, a phospholipid derived from soybean (e.g. Lipoid S100, Lipoid S75 or Lipoid S75-3N).

Preferred polar lipids (such as phospholipids) are those that swell to a measurable degree in water and/or those which are capable of spontaneous liposome formation.

If the polar (e.g. phospho-) lipid does not swell spontaneously in water, the skilled person will appreciate that it is nevertheless possible to obtain liposomes by adding a more polar, swellable (e.g. phospho-) lipid, such as an anionic (e.g. phospho-) lipid (e.g. phosphatidylglycerol).

Liposome formation may be performed at above about 0° C. (e.g. room temperature) if the phase transition temperature of the acyl chains (chain melting; gel-to-liquid crystals) is below the freezing point of water.

Whichever polar lipid substance (or combination thereof) is used, suitable total amounts/concentrations of lipid(s) that may be employed in preparation of a composition of the invention are in the range of about 10 mg/mL to about 120 mg/mL. Compositions of the invention that may be mentioned include those in which, when the polar lipid comprises phospholipid (whether in combination with another lipid or otherwise), the amount of phospholipid(s) in the composition is from about 10 (e.g. about 17, such as about 20) mg/mL to about 120 mg/mL, more preferably from about 25 (e.g. about 35) mg/mL to about 100 (e.g. about 70, such about 50, e.g. about 40) mg/mL. Typical ranges that may be mentioned include from about 25 (e.g. 27) mg/mL to about 50 mg/mL (e.g. 45 or, more particularly, 35 mg/mL). Further, the total amount of phospholipid (when the polar lipid comprises phospholipid) is preferably in the range from about 10 mg to about 80 mg (such as from about 17 (e.g. 20) mg to about 70 (e.g. 40) mg.

Compositions of the invention may also comprise an antioxidant, such as α-tocopherol, ascorbic acid, butylated hydroxyanisole, butylated hydroxytoluene, citric acid, fumaric acid, malic acid, monothioglycerol, propionic acid, propyl gallate, sodium ascorbate, sodium bisulfite, sodium metabisulfite, potassium metabisulfite, sodium sulfite, tartaric acid or vitamin E. Preferred antioxidants include butylated hydroxytoluene, α-tocopherol, ascorbic acid and butylated hydroxyanisole.

According to the invention a chelating agent may be used to reduce the metal ion catalysed oxidation of phospholipid and/or active ingredient(s). Examples of useful chelating agents are ethylenediaminetetraacetic acid (EDTA) and salts thereof (e.g. sodium or potassium EDTA), ethylenediaminetriacetic acid and diethylenetriaminepentaacetic acid (DTPA). It is also possible to use other agents that protect the composition of the invention and, in particular, any unsaturated fatty acid residues that may be present therein, from oxidation. Preferred chelating agents include EDTA and salts thereof.

The composition of the invention can comprise one or more preservatives. Examples of common preservatives for liquid pharmaceutical compositions are benzalkonium chloride, benzoic acid, butylated hydroxyanisole, butylparaben, chlorbutanol, ethylparaben, methylparaben, propylparaben, phenoxyethanol or phenylethyl alcohol. Preferred preservatives include benzalkonium chloride. Other preservatives that may be mentioned include sorbic acid.

In order to retain the composition of the invention at its application site it may also comprise viscosity-increasing agent such as, for instance, hydrophilic polymers like polyethyleneglycol, or crosslinked polyvinylpyrrolidone and/or cellulose derivatives such as hydroxypropylmethyl cellulose. Viscosity-increasing agents may also function as protective colloids to physically stabilise the composition of the invention prior to administration. Preferred protective colloids include hydroxypropylmethyl cellulose and, more particularly, polyethylene glycol.

Compositions of the invention may also comprise flavourings (e.g. lemon, menthol or peppermint powder) and/or sweeteners (e.g. neohesperidin).

Compositions of the invention may also comprise tonicity-modifying agents, such as sodium chloride, potassium chloride, glycerol, glucose, dextrose, sucrose, mannitol, etc.

Optional additives, including buffering agents, preservatives, viscosity-increasing agents, antioxidants, tonicity-modifying agents and chelating agents should be selected, in terms of their identity and the amounts employed, keeping in mind that their detrimental effect on liposome stability should be kept at a minimum. For a given agent this can be ascertained by simple experiments, which are well within the understanding of the skilled person. Suitable amounts of such ingredients are however in the range about 0.01 mg/mL to about 10 mg/mL. It is preferred that the compositions of the invention contain at least one preservative, antioxidant, chelating agent, buffering agent and/or viscosity-increasing agent. Suitable amounts of any/all of these optional additives include from about 0.02 to about 5 (e.g. about 3) mg/mL (e.g. from about 0.1 to about 2 mg/mL.

There is also provided a process for preparing compositions of the invention We have surprisingly found that liposomes may be prepared by direct swelling of the polar lipids in an aqueous medium without the addition of any other excipients such as charged lipids and/or surfactants etc., which are normally required.

According to a further aspect of the invention, there is provided a process for preparing a composition of the invention, which process comprises:

(a) mixing together (i) a polar lipid or a mixture of polar lipids that is/are swellable in aqueous media, (ii) an aqueous phase, and (in) an antiinflammatory and/or antihistaminic active ingredient; and
(b) homogenising the preparation.

Aqueous phases as employed in step (a) above include water, or water in which something else is dissolved (i.e. an aqueous solution). Aqueous solutions may comprise e.g. buffer (vide infra). Aqueous solutions may also comprise an antiinflammatory and/or antihistaminic active ingredient (i.e. component (iii) above), in which case the polar lipid, or mixture of polar lipids is/are added to an aqueous solution of an antiinflammatory and/or antihistaminic active ingredient in step (a) above.

Step (a) of the above-mentioned process is preferably carried out in the presence of suitable agitation (e.g. stirring).

Preferably the pH of the preparation is adjusted, for example prior to the homogenisation step (b) above, to a desired value within the range of from about pH 4 (e.g. 4.0) to about pH 8 (e.g. 8.0), preferably from about pH 5 (e.g. 5.0) to about pH 7 (e.g. 7.0), by adding an acid or a base (e.g. hydrochloric acid and/or sodium hydroxide at an appropriate concentration (e.g. 1M)).

Preferably water, saline or buffer solution is added, for example prior to the homogenisation step (b) above and/or after the pH adjusting step mentioned above, to the preparation to obtain a desired final batch volume.

Solutions/liquids may be purged with nitrogen or argon at a suitable stage in the above process, if and as appropriate.

In the context of the present invention, a lipid may be said to be swellable in aqueous media if, when placed in contact with such a medium, it swells to a measurable degree.

Buffers may preferably be added to the aqueous solution of drug (and/or drug may be added to an aqueous buffer solution) prior to the addition of lipid.

The formation of the liposomes of the invention may be facilitated by the spontaneous swelling of the polar lipid in water forming a lamellar liquid crystalline phase having a maximum water content of about 35% by weight or higher depending on the nature of the polar lipid. Depending on the lipid or lipid mixture used and other conditions, spontaneous formation of liposomes may be achieved when excess water is added to this lamellar phase. If spontaneous formation is not achieved, the formation of liposomes may be accomplished by the mechanical dispersion step (i.e. the homogenisation step (b) of the above process) of the lamellar liquid-crystalline phase in excess water.

Homogenisation/dispersion methods include vigorous mechanical mixing or high speed homogenisation, for instance by means of an Ultra Turrax® (Jankel & Kühnke, Germany). Shaking, vortexing and rolling may also be performed as part of the homogenisation step of the above process.

A homogeneous size distribution of the liposomes of the invention may be desirable and may be obtained by extrusion through a membrane filter, such as one made of polycarbonate, with a pore size of about 100 nm. Membrane filters may be procured from Avestin Inc., Canada.

A reduced average liposome size and narrowed liposome size distribution may preferably also be obtained when the liposomal dispersion is subjected to high-pressure homogenisation with a suitable homogeniser (Rannie APV, type 7.30 V H, Rannie A S, Denmark) at, for example, between about 300 bar and about 1000 bar, such as between about 400 bar and about 900 bar, e.g. about 500 to about 800 bar for between about 4 and about 8 (e.g. 7, such as 6) cycles.

We have found that the presence of certain active ingredients may result in a reduction of liposome size. Smaller liposomes are generally advantageous because they are more stable physically and, due to their higher surface area/volume ratio, are more easily resorbed by the mucosa.

We prefer that the diameter of liposomes in compositions of the invention is less than about 200 nm (e.g. between about 40 to about 100 nm), as measured by, for example, laser diffraction or dynamic light scattering, e.g. as described hereinafter.

Furthermore, the above-mentioned process for the preparation of compositions of the invention does not normally require conventional treatment with organic solvents such as chloroform or dichloromethane. However, if two or more membrane lipids are used it may be appropriate and/or necessary to treat them with organic solvent prior to the addition of the aqueous solvent. For example, the lipids may be dissolved in a volatile solvent or solvent mixture, such as chloroform or chloroform/methanol. The solution may then be deposited on the surfaces of a round-bottomed flask as the solvent is removed by rotary evaporation under reduced pressure. An excess volume of aqueous buffer containing the drug may then be added to the dry thin film of lipids, which may then be allowed to swell to form liposomes. In other cases, if the active ingredient is significantly insoluble in water and/or phospholipid, it may be necessary to dissolve it and the phospholipid in an organic solvent prior to addition of the aqueous phase. Again, organic solvent may be removed (e.g. in vacuo) prior to addition of the aqueous phase.

The compositions of the invention are useful in the treatment of any indication for which the relevant active ingredient is known to be effective, for example those specifically listed for the active ingredients in question in Martindale “The Complete Drug Reference”, 34^{th} Edition, Royal Pharmaceutical Society (2005). According to a further aspect of the invention, there is provided a method for the treatment of an inflammatory disorder (and/or migraine or pain (e.g. acute pain), as appropriate) comprising the administration of a pharmacologically-effective amount of a composition of the invention to a person suffering from or susceptible to that disorder.

For the avoidance of doubt, by “treatment” we include the therapeutic treatment, as well as the symptomatic treatment, the prophylaxis, or the diagnosis, of a condition.

Although compositions of the invention may be administered by any known route, including parenterally, topically and/or perorally, they may normally be administered transmucosally and, more particularly, nasally, ocularly and pulmonarily. For example, compositions of the invention may be administered by way of a nasal spray, nasal drops and/or eye drops. It is also possible to administer compositions of the invention as a fine mist to the lungs by nebulization. For nasal administration, any state-of-the-art device suitable for producing sprays of aqueous liposomal dispersions may be used.

Such formulations may be prepared in accordance with standard and/or accepted pharmaceutical practice.

Wherever the word “about” is employed herein in the context of dimensions (e.g. pH values, sizes, temperatures, pressures, etc.) and amounts (e.g. amounts, weights and/or concentrations of individual constituents in a composition or a component of a composition, proportions of drug inside/outside the liposomal structures, absolute doses of active ingredient, etc.), it will be appreciated that such variables are approximate and as such may vary by ±10%, for example ±5% and preferably ±2% (e.g. ±1%) from the numbers specified herein.

The compositions of the invention, and the above-mentioned process that may be employed for their preparation, have the advantages that are mentioned hereinbefore. In particular, compositions of the invention may reduce the incidence of inconvenient side-effects (and in particular irritation) that are often observed with e.g. nasally-administered formulations.

Compositions of the invention are easy to manufacture and enable the production of liposomal-based formulations that are in a ready-to-use form, avoiding the need for reconstitution prior to administration.

Compositions of the invention may also have the advantage that they may be prepared using established pharmaceutical processing methods and employ materials that are approved for use in foods or pharmaceuticals or of like regulatory status.

Compositions of the invention may also have the advantage that they may be more efficacious than, be less toxic than, be longer acting than, be more potent than, produce fewer side effects than, be more easily absorbed than, and/or have a better pharmacokinetic profile than, and/or have other useful pharmacological, physical, or chemical properties over, pharmaceutical compositions known in the prior art, whether for use in the treatment of rhinitis or otherwise.

The invention is illustrated by way of the following examples.

General procedure. For weights and volumes reference is made to the tables below. A buffer solution is prepared by dissolving anhydrous citric acid and solid sodium hydroxide in 160 mL water (80% of the total batch volume) in a 200 mL volumetric flask. The weighed amount of active agent is added and dissolved by stirring with a magnetic stirrer. The phospholipid is separately weighed and added to the solution. Stirring is continued until a well dispersed suspension has formed, the pH of which is adjusted to pH 5.0±0.1 with 1.0 M NaOH and/or 1.0 M HCl. The volume of the preparation is then brought to the final batch volume of 200 mL. The preparation is transferred to a high pressure homogeniser (Rannie APV, type 7.30 V H, Ramie A S, Denmark) and homogenised at 500-800 bar for 5 cycles. Aliquots of the thus obtained composition are removed from the collecting vessel and transferred to glass vials.

The above procedure is/was employed in order to prepare final compositions as outlined by Examples 1 to 8 below. Where appropriate, the quantities of the components are/were scaled up appropriately (e.g. in the case of Examples 1 to 8, multiplied by 200). The procedure for Example 9 is described separately below.

### EXAMPLE 1

| Budesonide | 1.3 | mg |
|---|---|---|
| Phospholipid (soybean; Lipoid S100; Lipoid GmbH, | 35.0 | mg |
| Germany) | | |
| Benzalkonium chloride | 0.1 | mg |
| Butylated hydroxytoluene (BHT) | 0.1 | mg |
| Hydroxypropylmethylcellulose (Metolose 60SH-50) | 10 | mg |
| Citric acid | 19.2 | mg |
| Sodium hydroxide | 8.4 | mg |
| 1 M HCl and/or 1 M NaOH | to pH 5.5 | |
| Water for injection | to 1 | mL |

### EXAMPLE 2

| Fluticasone propionate | 0.5 | mg |
|---|---|---|
| Phospholipid (soybean; Lipoid S100; Lipoid GmbH, | 17.5 | mg |
| Germany) | | |
| Phospholipid (DMPC; Lipoid GmbH, Germany) | 17.5 | mg |
| Benzalkonium chloride | 0.1 | mg |
| Butylated hydroxytoluene (BHT) | 0.1 | mg |
| Citric acid | 19.2 | mg |
| Sodium hydroxide | 8.4 | mg |
| 1 M HCl and/or 1 M NaOH | to pH 5.5 | |
| Water for injection | to 1 | mL |

### EXAMPLE 3

| Zileuton | 200.0 | mg |
|---|---|---|
| Phospholipid (soybean; Lipoid S100; Lipoid GmbH, | 23.3 | mg |
| Germany) | | |
| Phospholipid (DMPC; Lipoid GmbH, Germany) | 11.7 | mg |
| Benzalkonium chloride | 0.1 | mg |
| Butylated hydroxytoluene (BHT) | 0.1 | mg |
| Citric acid | 19.2 | mg |
| Sodium hydroxide | 8.4 | mg |
| 1 M HCl and/or 1 M NaOH | to pH 5.5 | |
| Water for injection | to 1 | mL |

### EXAMPLE 4

| Azelastine | 0.9 | mg |
|---|---|---|
| Phospholipid (soybean; Lipoid S100; Lipoid GmbH, | 23.3 | mg |
| Germany) | | |
| Phospholipid (DMPC; Lipoid GmbH, Germany | 11.7 | mg |
| Benzalkonium chloride | 0.1 | mg |
| Butylated hydroxytoluene (BHT) | 0.1 | mg |
| Polyethylene glycol (Macrogol 6000) | 10 | mg |
| Citric acid | 19.2 | mg |
| Sodium hydroxide | 8.4 | mg |
| 1 M HCl and/or 1 M NaOH | to pH 5.5 | |
| Water for injection | to 1 | mL |

| Montelukast | 25 | mg |
|---|---|---|
| Phospholipid (soybean; Lipoid S100; Lipoid GmbH, | 29.2 | mg |
| Germany) | | |
| Phospholipid (DMPC; Lipoid GmbH, Germany | 5.8 | mg |
| Benzalkonium chloride | 0.1 | mg |
| Butylated hydroxytoluene (BHT) | 0.01 | mg |
| Povidone | 1.0 | mg |
| Citric acid | 19.2 | mg |
| Sodium hydroxide | 8.4 | mg |
| 1 M HCl and/or 1 M NaOH | to pH 5.5 | |
| Water for injection | to 1 | mL |

### EXAMPLE 6

| Etoricoxib | 150.0 | mg |
|---|---|---|
| Phospholipid (soybean; Lipoid S100; Lipoid GmbH, | 23.3 | mg |
| Germany) | | |
| Phospholipid (DMPC; Lipoid GmbH, Germany | 11.7 | mg |
| Benzalkonium chloride | 1.0 | mg |
| Butylated hydroxytoluene (BHT) | 0.1 | mg |
| Hydroxypropylmethylcellulose (Metolose 60SH-50) | 5.0 | mg |
| Citric acid | 19.2 | mg |
| Sodium hydroxide | 8.4 | mg |
| 1 M HCl and/or 1 M NaOH | to pH 5.5 | |
| Water for injection | to 1 | mL |

### EXAMPLE 7

| Budesonide | 1.3 | mg |
|---|---|---|
| Phospholipid (soybean; Lipoid S100; Lipoid GmbH, | 35.0 | mg |
| Germany) | | |
| Benzalkonium chloride | 0.2 | mg |
| Butylated hydroxytoluene (BHT) | 0.2 | mg |
| Citric acid | 19.2 | mg |
| Sodium hydroxide | 8.4 | mg |
| 1 M HCl and/or 1 M NaOH | to pH 5.0 | |
| Water for injection | to 1 | mL |

### EXAMPLE 8

| Fluticasone propionate | 0.5 | mg |
|---|---|---|
| Phospholipid (soybean; Lipoid S100; Lipoid GmbH, | 27.0 | mg |
| Germany) | | |
| Phospholipid (DMPC; Lipoid GmbH, Germany) | 8.0 | mg |
| Sorbic acid | 1.0 | mg |
| Na EDTA | 0.1 | mg |
| Butylated hydroxytoluene (BHT) | 0.2 | mg |
| Citric acid | 19.2 | mg |
| Sodium hydroxide | 8.4 | mg |
| 1 M HCl and/or 1 M NaOH | to pH 5.0 | |
| Water for injection | to 1 | mL |

### EXAMPLE 9

The commercially available nasal antihistamine azelasine (registered under trade names such as Azelvin®, Azosin®, Astelin®, Lastin® and Rhinolast® was formulated using the quantities and steps outlined below.
1. 160 mL azelastine solution for nasal administration (Lastin®) containing 0.9 mg/mL azelastine was transferred into a 200 mL volumetric flask.
2. 7 g Soy bean phospholipid (Lipoid S100; Lipoid GmbH, Germany) was added and the mixture was allowed to swell overnight.
3. The volume was brought to 200 mL by the addition of more azelastine solution (see step 1 above).
4. The pH was checked.
5. The solution was homogenised for 7 cycles at 800 bar as described in the general procedure above.

1. A homogeneous pharmaceutical composition for the treatment of an inflammatory disorder comprising an antiinflammatory and/or antihistaminic active ingredient, a polar lipid liposome and a pharmaceutically-acceptable aqueous carrier, provided that the active ingredient is not cetirizine.

2. A composition as claimed in claim 1, which further includes a pharmaceutically-acceptable buffer capable of providing a pH of from about pH 4 to about pH 8.

3. A composition as claimed in claim 2, wherein the pH range is about pH 5 to about pH 7.

4. A composition as claimed in claim 2, wherein the buffer is a phosphate, citrate or acetate buffer.

5. A composition as claimed in claim 4, wherein the buffer is disodium phosphate, dipotassium phosphate, sodium dihydrogen phosphate, potassium dihydrogen phosphate, phosphoric acid plus base, sodium citrate, citric acid plus base, sodium acetate or acetic acid plus base.

6. A composition as claimed in claim 2, wherein the quantity of buffer is in the range of about 1 mg/mL to about 30 mg/mL.

7. A composition as claimed in claim 1 wherein the active ingredient is an antihistamine.

8. A composition as claimed in claim 7 wherein the antihistamine is selected from acrivastine, alimemazine, anatazoline, astemizole, azatadine, azelastine, bamipine, bepotastine, bromazine, bromopheniramine, buclizine, carbinoxamine, chlorocyclizine, chloropyramine, chlorophenamine, cinnarizine, clemastine, clemizole, clocinizine, cyclizine, cyproheptadine, deptropine, desloratadine, dexchlorpheniramine, dimenhydrinate, dimetindene, dimetotiazine, diphenhydramine, piphenylpyraline, doxylamine, ebastine, embramine, emedastine, epinastine, fexofenadine, flunarizine, homochlorocyclizine, hydroxyzine, isothipendyl, levocarbastine, loratidine, mebhydroline, meclozine, mepyramine, mequitazine, methdilazine, mizolastine, niaprazine, olopatadine, oxatomide, oxomemazine, phenindamine, pheniramine, phenyltoloxamine, pimethixene, pipinhydrinate, promethazine, propiomazine, quifenadine, ruputadine, setastine, terfenadine, thenyidiamine, thiethylperazine, thonzylamine, tolpropaminr, trimethobenzamine, tripelennamine, triprolidine, tritoqualine and a pharmaceutically-acceptable salt of any of these compounds.

9. A composition as claimed in claim 1 wherein the active ingredient is an antiinflammatory agent.

10. A composition as claimed in claim 9 wherein the antiinflammatory agent is a steroid.

11. A composition as claimed in claim 10 wherein the steroid is selected from alclometasone, beclometasone, betamethasone, budesonide, ciclesonide, clobetasol, clobetasone, deflazacort, dexamethasone, diflucortolone valerate, fluocinolone acetonide, fluocinonide, fluocortolone, fluprednidene, fluorometholone, fluticasone, halcinonide, hydrocortisone, methylprednisolone, mometasone, prednisolone, rimexolone, triamcinolone and a pharmaceutically-acceptable salt of any of these compounds.

12. A composition as claimed in claim 9 wherein the antiinflammatory agent is a non-steroidal antiinflammatory drug.

13. A composition as claimed in claim 12 wherein the non-steroidal antiinflammatory drug is a PDE4 inhibitor.

14. A composition as claimed in claim 12 wherein the non-steroidal antiinflammatory drug is a leukotriene modifier.

15. A composition as claimed in claim 14 wherein the leukotriene modifier is a 5-lipoxygenase inhibitor.

16. A composition as claimed in claim 14 wherein the leukotriene modifier is a FLAP inhibitor.

17. A composition as claimed in claim 14 wherein the leukotriene modifier is a CysLT antagonist.

18. A composition as claimed in claim 1, wherein the polar lipid is of a natural origin, is of a synthetic/semi-synthetic origin, or comprises a mixture of the two.

19. A composition as claimed in claim 1, wherein the polar lipid comprises or consists of a phospholipid or a mixture of phospholipids.

20. A composition as claimed in claim 19, wherein the phospholipid comprises one that is based on phosphatidylcholine, phosphatidylglycerol, phosphatidylinositol, phosphatidic acid, phosphatidylserine or a mixture thereof.

21. A composition as claimed in claim 20, wherein the phospholipid comprises one that is represented by the general formula I, 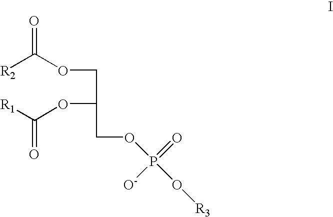
wherein R₁ and R₂ independently represent a saturated or unsaturated, branched or straight chain alkyl group having between 7 and 23 carbon atoms and R₃ represents an amide or ester bonding group.

22. A composition as claimed in claim 21, wherein the amide or ester bonding group is —CH₂—CH(OH)—CH₂OH, —CH₂—CH₂—N(CH₃)₃, —CH₂—CH₂—NH₂, —H or —CH₂—CH(NH₂)—COOH.

23. A composition as claimed in claim 22, wherein the phospholipid comprises a membrane lipid derived from soybean.

24. A composition as claimed in claim 23, wherein the phospholipid comprises Lipoid S75, Lipoid S100 and/or Lipoid S75-3N.

25. A composition as claimed in claim 24, wherein the phospholipid comprises dilaurylphosphatidylcholine, dimyristolphosphatidyl-choline, dipalmitoylphosphatidylcholine, dilaurylphosphatidylglycerol, dimyristolphosphatidylglycerol, dioleoylphosphatidylcholine or dioleoylphosphatidylglycerol.

26. A composition as claimed in claim 18, wherein the polar lipid comprises or consists of a glycolipid or a mixture of glycolipids.

27. A composition as claimed in claim 26, wherein the glycolipid comprises a glycoglycerolipid.

28. A composition as claimed in claim 27, wherein the glycoglycerolipid comprises a galactoglycerolipid.

29. A composition as claimed in claim 27, wherein the glycoglycerolipid comprises a digalactosyldiacylglycerol of the general formula II, 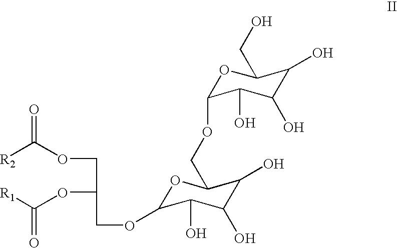
wherein R₁ and R₂ are as defined in claim 21.

30. A composition as claimed in claim 26, wherein the glycolipid comprises digalactosyldiacylglycerol.

31. A composition as claimed in claim 26, wherein the glycolipid comprises a glycosphingolipid.

32. A composition as claimed in claim 31, wherein the glycosphingolipid comprises a monoglycosylsphingoid, an oligoglycosylsphingoid, an oligoglycosylceramide, a monoglycosylceramide, a sialoglycosphingolipid, a uronoglycosphingolipid, a sulfoglycosphingolipid, a phosphoglycosphingolipid, a phosphonoglycosphingolipid, a ceramide, a monohexosylceramide, a dihexosylceramide, a sphingomyelin, a lysosphingomyelin, a sphingosine or a mixture thereof.

33. A composition as claimed in claim 32, wherein the glycosphingolipid comprises sphingomyelin or a product derived therefrom.

34. A composition as claimed in claim 26, wherein the glycolipid comprises a glycophosphatidylinositol.

35. A composition as claimed in claim 1, wherein the amount of polar lipid substance that is used is in the range of about 10 mg/mL to about 120 mg/mL.

36. A composition as claimed in claim 1, wherein the amount of phospholipid in the composition is from about 17 mg/mL to about 70 mg/mL.

37. A composition as claimed in claim 36, wherein the amount is from about 20 mg/mL to about 40 mg/mL.

38. A composition as claimed in claim 1, which further comprises an antioxidant.

39. A composition as claimed in claim 38, wherein the antioxidant is I-tocopherol, ascorbic acid, butylated hydroxyanisole, butylated hydroxytoluene, citric acid, fumaric acid, malic acid, monothioglycerol, propionic acid, propyl gallate, sodium ascorbate, sodium bisulfite, sodium metabisulfite, potassium metabisulfite, sodium sulfite, tartaric acid and/or vitamin E.

40. A composition as claimed in claim 1, which further comprises a chelating agent.

41. A composition as claimed in claim 40, wherein the chelating agent is ethylenediaminetetraacetic acid (and/or a salt thereof, ethylenediaminetriacetic acid and/or diethylenetriaminepentaacetic acid.

42. A composition as claimed in claim 1, which further comprises a preservative.

43. A composition as claimed in claim 42, wherein the preservative is benzalkonium chloride, benzoic acid, butylated hydroxyanisole, butylparaben, chlorbutanol, ethylparaben, methylparaben, propylparaben, phenoxyethanol and/or phenylethyl alcohol.

44. A composition as claimed in claim 1, which further comprises a viscosity-increasing agent.

45. A composition as claimed in claim 44, wherein the viscosity-increasing agent is polyethyleneglycol, crosslinked polyvinylpyrrolidone and/or hydroxypropylmethyl cellulose.

46. A composition as claimed in claim 1, wherein the diameter of the liposomes is less than about 200 nm.

47. A composition as claimed in claim 46, wherein the diameter is between about 40 nm and about 100 nm.

48. A process for the preparation of a composition as claimed in claim 1, which process comprises:
(a) mixing together (i) a polar lipid or a mixture of polar lipids that is/are swellable in aqueous media, (ii) an aqueous phase, and (iii) an antiinflammatory and/or antihistaminic active ingredient; and
(b) homogenising the preparation.

49. A process as claimed in claim 48, wherein the polar lipid, or mixture of polar lipids is/are added to an aqueous solution of an antiinflammatory and/or antihistaminic active ingredient in step (a).

50. A process as claimed in claim 49, wherein, prior to the homogenisation step, the pH is adjusted to the desired value by adding an acid or a base.

51. A process as claimed in claim 48, wherein, prior to the homogenisation step, water, saline or buffer solution is added to the preparation to obtain a desired final batch volume.

52. A process as claimed in claim 51, wherein the addition of water, saline or buffer takes place after the pH adjusting step.

53. A process as claimed in claim 48, wherein at least one of the solutions/liquids is/are purged with nitrogen and/or argon.

54. A process as claimed in claim 49, wherein the aqueous solution of active ingredient is formed either by adding buffer to an aqueous solution of the active ingredient, or adding the active ingredient to an aqueous buffer solution, prior to the addition of lipid.

55. A process as claimed in claim 48, wherein, if a mixture of polar lipids is used, it is pre-treated with organic solvent.

56. A process as claimed in claim 48, wherein, if the active ingredient is significantly insoluble in water, it is pre-treated with organic solvent (in combination with the lipid).

57. A process as claimed in claim 48, wherein the homogenisation step (b) comprises vigorous mechanical mixing, high speed homogenisation, shaking, vortexing and/or rolling.

58. A process as claimed in claim 48, which comprises an additional liposome size-reduction step.

59. A process as claimed in claim 58, wherein the size-reduction step comprises extrusion through a membrane filter.

60. A process as claimed in claim 48, wherein the homogenisation step and/or size-reduction step comprises high-pressure homogenisation.

61. A homogeneous pharmaceutical composition for the treatment of an inflammatory disorder comprising an antiinflammatory and/or antihistaminic active ingredient, a polar lipid liposome and a pharmaceutically-acceptable aqueous carrier, provided that the active ingredient is not cetirizine, obtainable by a process comprising or consisting essentially of:
(a) mixing together (i) a polar lipid or a mixture of polar lipids that is/are swellable in aqueous media, (ii) an aqueous phase, and (iii) the antiinflammatory and/or antihistaminic active ingredient; and
(b) homogenising the preparation.

62. A composition as claimed in claim 61, wherein, in the process, the polar lipid, or mixture of polar lipids is/are added to an aqueous solution of an antiinflammatory and/or antihistaminic active ingredient in step (a).

63. A composition as claimed in claim 61, wherein, in the process, prior to the homogenisation step, the pH is adjusted to the desired value by adding an acid or a base.

64. A composition as claimed in claim 63, wherein, in the process, prior to the homogenisation step, water, saline or buffer solution is added to the preparation to obtain a desired final batch volume.

65. A composition as claimed in claim 64, wherein the addition of water, saline or buffer takes place after the pH adjusting step.

66. A composition as claimed in claim 61, wherein, in the process, at least one of the solutions/liquids is/are purged with nitrogen and/or argon.

67. A composition as claimed in claim 62, wherein, in the process, the aqueous solution of the active ingredient is formed either by adding buffer to the aqueous solution of an active ingredient, or adding an active ingredient to an aqueous buffer solution, prior to the addition of lipid.

68. A composition as claimed in claim 68, wherein, in the process, if a mixture of polar lipids is used, it is pre-treated with organic solvent.

69. A composition as claimed in claim 61, wherein, in the process, if the active ingredient is significantly insoluble in water, it is pre-treated with organic solvent (in combination with the lipid).

70. A composition as claimed in claim 61, wherein, in the process, the homogenisation step (b) comprises vigorous mechanical mixing, high speed homogenisation, shaking, vortexing and/or rolling.

71. A composition as claimed in claim 61, which comprises, in the process, an additional liposome size-reduction step.

72. A composition as claimed in claim 71, wherein the size-reduction step comprises extrusion through a membrane filter.

73. A composition as claimed in claim 61, wherein, in the process, the homogenisation step and/or size-reduction step comprises high-pressure homogenisation.

74. A composition as claimed in claim 1, for use in medicine.

75. A method for the treatment of an inflammatory disorder comprising the administration of a composition as claimed in claim 1, to a person suffering from or susceptible to that disorder.

76. (canceled)

77. A method as claimed in claim 75, wherein the inflammatory disorder is rhinitis.

78. A method as claimed in claim 75, wherein the inflammatory disorder is asthma.

79. A method as claimed in claim 75, wherein the inflammatory disorder is inflammatory pain.

80. A method as claimed in claim 75 wherein the composition is administered nasally.

81. A composition as claimed in claim 1 wherein the active ingredient is an anti-migraine compound.

82. A composition as claimed in claim 81 wherein the anti-migraine compound is selected from almotriptan, alpiropride, dihydroergotamine, eletriptan, ergotamine, feverfew, frovatriptan, iprazochrome, methysergide, naratriptan, pizotifen, rizatriptan, sumatriptan, zolmitriptan and a pharmaceutically-acceptable salt of any of these compounds.

83. A composition as claimed in claim 1 wherein the active ingredient is an opioid or an analogue thereof.

84. A composition as claimed in claim 83 wherein the opioid or analogue thereof is selected from alfentanil, anileridine, bezitramide, buprenorphine, butorphanol, carfentanil, codeine, dextromoramide, dextropropoxyphene, dezocine, diamorphine, dihydrocodeine, dipipanone, embutramide, ethoheptazine, ethylmorphine, etorphine, fentanyl, hydrocodone, hydromorphone, ketobemidone, levacetylmethadol, levomethadone, levophanol, lofexidine, meptazinol, methadone, morphine, nalbuphine, naltrexone, nicomorphine, opium, oxycodone, oxymorphone, papaveretum, pentazocine, pethidine, phenazocine, phenoperidine, pholcodine, piritramide, remifentanil, sufentanil, tilidine, tramadol and a pharmaceutically-acceptable salt of any of these compounds.

85. A method for the treatment of migraine comprising the administration of a composition as claimed in claim 81 to a person suffering from or susceptible to that disorder.

86. A method for the treatment of pain comprising the administration of a composition as claimed in claim 83 to a person suffering from or susceptible to that disorder.
